(19) United States
(12) Patent Application Publication
(10) Pub. No.: US 2023/0181540 A1
(43) Pub. Date: Jun. 15, 2023

(54) COMPOSITIONS AND METHODS FOR ACTIVATING SIGNALING THROUGH THE CB2 CANNABINOID RECEPTOR FOR TREATING AND PREVENTING LYSOSOMAL STORAGE DISEASES AND DISORDERS

(71) Applicant: Wylder Nation Foundation, Scottsdale, AZ (US)

(72) Inventors: Edward H. SCHUCHMAN, Haworth, NJ (US); Calogera M. SIMONARO, Haworth, NJ (US)

(21) Appl. No.: 17/925,188

(22) PCT Filed: May 20, 2021
(86) PCT No.: PCT/US2021/033333

§ 371 Date: Nov. 14, 2022

Related U.S. Application Data
(60) Provisional application No. 63/027,817, filed on May 20, 2020.

(51) Int. Cl. A61K 31/426; A61K 31/4155; A61K 31/421; A61K 31/429; A61P 3/00

(52) U.S. Cl. CPC A61K 31/426; A61K 31/421; A61K 31/429; A61K 31/4155; A61P 3/00

(57) ABSTRACT

The present invention provides, inter alia, compositions, methods, and diagnostics for using CB2 cannabinoid receptor agonists for treating and preventing lysosomal storage disorders.

### CROSS-REFERENCE

This application claims the benefit of U.S. Provisional Application No. 63/027,817, filed May 20, 2020, which application is incorporated herein by reference in its entirety.

### FIELD OF THE INVENTION

The present disclosure relates to the treatment and prevention of lysosomal storage diseases and disorders. In particular, the present disclosure relates to compositions and methods for activating the CB2 cannabinoid receptor for treating or preventing lysosomal storage diseases and disorders.

### BACKGROUND OF THE INVENTION

Lysosomal storage disorders are a group of approximately 60 genetic orphan diseases caused by specific gene/protein defects (mostly in lysosomal enzymes) and the resultant accumulation of specific substrates, but they share several common pathogenic mechanisms. For example, accumulation of the primary substrate in any individual lysosomal disorder will eventually lead to the overall dysfunction of the lysosomal system, including dysfunction of other enzymes within the lysosomes, and the eventual buildup of many secondary substrates. Many of these accumulating secondary substrates are pathogenic and common among the lysosomal disorders, including the sphingolipids, ceramide, and sphingosine, as well as glycosaminoglycans (GAGs), glycogen and others. Since ceramide is the precursor for all other complex sphingolipids, including sphingomyelin and gangliosides, these lipids often accumulate in many of the lysosomal diseases, and is particularly pathogenic. Thus, in many, if not all, instances the pathology of any given lysosomal disease is due not only to the accumulation of the primary substrate, but to the buildup of these additional substrates as well, and the eventual disruption of the cellular, lysosomal apparatus, induction of chronic inflammation, and cell death.

Various treatment strategies have been developed for these diseases, including enzyme replacement therapies, gene therapies, and substrate reduction therapies. To address the common pathogenic mechanisms in the lysosomal storage diseases and disorders, several studies also have been undertaken using generic anti-inflammatory therapies in mouse models of these diseases; however, the results to date have resulted in limited or partial success. For example, in a mouse model of Sanfilippo disease (mucopolysaccharidosis IIIA), chronic treatment for 6 months with high doses of aspirin (5-10 fold higher than is used in humans) has been reported to normalize expression of some inflammation-related genes, suggesting a potential benefit; however no data regarding clinical impact or reduction in substrate storage (i.e., GAGs) was shown, thus, calling into question the relevance of the results (Arfi A, et al., Mol Genet Metab. 2011 May; 103(1): 18-25). In another study, aspirin failed to show treatment efficacy in a mouse model of Niemann-Pick diseases type C1 (NPC1); for example, high doses of the NSAID ibuprofen was reported to prolong the lifespan of the mice by about two weeks and to modestly slow the onset of clinical signs (Smith D. et. al, Neurobiol Dis. 2009 Nov;36(2):242-51). However, the dose used for the ibuprofen in this Smith et al. study was significantly higher than that which is used in humans. In addition, chronic use of each of ibuprofen and aspirin is associated with organ toxicities that would likely be particularly pronounced in LSD patients given that Smith et al., also demonstrated that NPC mice tolerated liver metabolized drugs poorly (Smith (2009)). Prednisolone has also been reported to slow CNS disease progression in a mouse model of mucopolysaccharidosis IIIB, suggesting anti-inflammatory intervention may be beneficial in this disease. However, chronic treatment with corticosteroids can have severe side effects including glaucoma, cataracts, hypertension, high blood sugar (which can worsen or trigger diabetes), osteoporosis, weight gain, etc., thus, it is unclear whether such a treatment would provide a viable long-term option for LSD patients. Another anti-inflammatory drug, pentosan polysulfate (PPS), has been studied in several LSD animal models and some clinical and pathological benefits have been shown (Frohbergh (2014), Simonaro (2016), Guo (2018). However, PPS does not penetrate the blood brain barrier and thus the impact on the CNS is likely to be limited. In fact, in a mouse model of MPS IIIA, while some improvements in CNS pathology were noted, this was attributed to systemic effects that limited the infiltration of immune cells into the brain. Thus, new drugs that can directly act in the CNS are needed for these diseases. Moreover, chronic use of PPS can lead to GI toxicities and coagulation abnormalities that may limit its usefulness in these life-long diseases.

Accordingly, there is a great need for new therapies that can treat or prevent the common and devastating symptoms and mortality associated with lysosomal storage disorders, particularly in the brain but in other organs as well, and a method of treating many or all of these diverse disorders would meet a long felt need in the art.

The present invention provides such a universal treatment for lysosomal storage diseases and disorders.

### SUMMARY OF THE INVENTION

The present disclosure relates to, inter alia, compositions and methods for treating and preventing lysosomal storage diseases and disorders. In particular embodiments, the present disclosure relates to compositions and methods for activating the CB2 cannabinoid receptor for treating or preventing lysosomal storage diseases and disorders. The present invention is based in part on the surprising discovery that CB2 cannabinoid receptor expression is increased significantly in the periphery and brain of subjects having the lysosomal storage diseases MPS IIIA and Farber disease, which have diverse molecular pathogenesis, but each exhibit aberrant substrate storage (GAGs and lipids, respectively), and resultant inflammation. This is a critical observation, as activation of the CB2 receptor has been associated with reduced inflammation and reduced cell death in some disease models. Thus, overexpression of this receptor in lysosomal storage diseases and disorders makes it an ideal therapeutic target in these conditions. Indeed, we show herein that the selective activation of the CB2 receptor in a well-characterized mouse model for Farber Disease (acid ceramidase deficiency) results in vast improvements in overall survival and weight. To date, we are not aware of any reports demonstrating elevated CB2 expression in lysosomal disease models or of any reports suggesting that activation of the CB2 receptor might present a viable means for reducing inflammation in subjects suffering from these diseases or disorders. Thus, the present invention provides, inter alia, compositions and methods for activating CB2 signaling as a treatment for lysosomal storage diseases and disorders.

In some embodiments, the present disclosure includes a method for reducing inflammation in a subject that has a lysosomal storage disease or disorder comprising, selectively or specifically activating the CB2 cannabinoid receptor in the subject. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Spingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the transport diseases is selected from Cystinosis and Salla Disease. In some embodiments, the neuronal ceroid lipofuscinosis is Batten’s disease. In some embodiments, the lysosomal storage disease or disorder is Farber Disease, or mucopolysaccharidosis type IIIA (MPS IIIA). In some embodiments, the inflammation is modulated in the subject’s periphery. In some embodiments, the inflammation is modulated by activating the CB2 receptor on peripheral macrophages in the subject. In some embodiments, the inflammation is modulated in the subject’s CNS. In some embodiments, the subject’s CNS by activating the CB2 receptor on microglia in the subject. In some embodiments, the inflammation is modulated in the subject’s CNS by inhibiting microglia activation. In some embodiments, the CB2 cannabinoid receptor is activated by a selective or specific agonist of the CB2 cannabinoid receptor. In some embodiments, the selective or specific CB2 agonist is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor selected from HU-308, JWH-015, GW-405833 (L-768242), AM-1241, JWH-133, GW-842166X, and a CB2 agonist compound disclosed herein. In some embodiments, the CB2 activation treats the lysosomal storage disease or disorder or alleviates one or more symptom of the lysosomal storage disease or disorder. In some embodiments, the method prevents neuronal death associated with the lysosomal storage disease or disorder. the method improves the subject’s cognition, learning and/or memory.

In some embodiments, the present disclosure includes a method of treating a subject that has a lysosomal storage disease or disorder comprising, administering to the subject a selective or specific agonist of the CB2 cannabinoid receptor. In some embodiments, the present disclosure includes a method of inhibiting a loss of motor skills, cognitive decline, and/or systemic organ pathology associated with a lysosomal storage disease or disorder comprising, administering to the subject a selective or specific agonist of the CB2 cannabinoid receptor. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the transport diseases is selected from Cystinosis and Salla Disease. In some embodiments, the neuronal ceroid lipofuscinosis is Batten’s disease. In some embodiments, the lysosomal storage disease or disorder is Farber Disease or mucopolysaccharidosis type IIIA (MPS IIIA). In some embodiments, the selective or specific agonist of the CB2 cannabinoid receptor is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor selected from HU-308, JWH-015, GW-405833 (L-768242), AM-1241, JWH-133, GW-842166X, and a CB2 agonist compound disclosed herein. In some embodiments, the method treats the lysosomal storage disease or disorder or results in the improvement of one or more symptoms associated with the lysosomal storage disease or disorder. In some embodiments, the method prevents neuronal death associated with the lysosomal storage disease or disorder. In some embodiments, the method improves the subject’s cognition. In some embodiments, the method improves the subject’s learning and/or memory. In some embodiments, the method reduces inflammation levels in the subject. In some embodiments, the method treats the brain or CNS of the subject. In some embodiments, the method activates CB2 on microglia in the brain of the subject. In some embodiments, the method reduces activation of microglia in the brain of the subject. In some embodiments, the method treats the reticuloendothelial system. In some embodiments, the method treats the skeletal system. In some embodiments, the method results in reduced inflammation levels in an organ other than the brain. In some embodiments, the organ other than the brain is selected from the liver, spleen, lungs, adrenal gland, kidney, heart, articular cartilage, articular joint space, and bone marrow. In some embodiments, the lifespan of the subject is increased as compared to the expected lifespan of an individual with the same lysosomal storage disease or disorder that has not been administered the selective agonist of the CB2 cannabinoid receptor. In some embodiments, the method further comprises administering one or more additional therapeutic agents. In some embodiments, the additional therapeutic agent comprises an enzyme replacement therapy, gene therapy, substrate reduction therapy, chaperone therapy, or a small molecule therapy. In some embodiments, the method further comprises activating the CB1 cannabinoid receptor. In some embodiments, the method further comprises administering a direct or indirect agonist of the CB1 cannabinoid receptor, wherein the indirect agonist is optionally a FAAHi.

In some embodiments, the present disclosure includes a method of inhibiting macrophage activation in a subject with a lysosomal storage disease or disorder comprising administering to the subject a selective or specific agonist of the CB2 cannabinoid receptor. In some embodiments, the macrophage is a peripheral macrophage in the subject. In some embodiments, the macrophage is a microglia in the central nervous system (CNS) of the subject. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the transport diseases is selected from Cystinosis and Salla Disease. In some embodiments, the neuronal ceroid lipofuscinosis is Batten’s disease. In some embodiments, the lysosomal storage disease or disorder is Farber Disease or mucopolysaccharidosis type IIIA (MPS IIIA). In some embodiments, the selective or specific agonist of the CB2 cannabinoid receptor is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor is selected from HU-308, JWH-015, GW-405833 (L-768562), AM-1561, JWH-133, GW-842166X, and a CB2 agonist compound disclosed herein. In some embodiments, the method treats the lysosomal storage disease or disorder or results in the improvement of one or more symptoms associated with the lysosomal storage disease or disorder. In some embodiments, the method prevents neuronal death associated with the lysosomal storage disease or disorder. In some embodiments, the method improves the subject’s cognition. In some embodiments, the method improves the subject’s learning and/or memory. In some embodiments, the method reduces inflammation levels in the subject. In some embodiments, the method activates CB2 in the brain or CNS of the subject. In some embodiments, the method activates CB2 on microglia in the brain of the subject. In some embodiments, the method treats the reticuloendothelial system. In some embodiments, the method treats the skeletal system. In some embodiments, the method results in reduced inflammation levels in an organ other than the brain. In some embodiments, the organ other than the brain is selected from the liver, spleen, lungs, adrenal gland, kidney, heart, articular cartilage, articular joint space, and bone marrow. In some embodiments, the lifespan of the subject is increased as compared to the expected lifespan of an individual with the same lysosomal storage disease or disorder that has not been administered the selective agonist of the CB2 cannabinoid receptor. In some embodiments, the method further comprises administering one or more additional therapeutic agents. In some embodiments, the additional therapeutic agent comprises an enzyme replacement therapy, gene therapy, substrate reduction therapy, chaperone therapy, or a small molecule therapy. In some embodiments, the method further comprises administering a direct or indirect agonist of the CB1 cannabinoid receptor, wherein the indirect agonist is optionally a FAAHi. A method of reducing at least one inflammatory cytokine or chemokine in a subject with a lysosomal storage disease or disorder comprising selectively or specifically activating the CB2 cannabinoid receptor. In some embodiments, the inflammatory cytokine or chemokine is selected from MCP-1, MIP1 alpha, TNF alpha, IL-1beta, IL-8, GM-CSF. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the transport diseases is selected from Cystinosis and Salla Disease. In some embodiments, the neuronal ceroid lipofuscinosis is Batten’s disease. In some embodiments, the lysosomal storage disease or disorder isFarber Diseaseor mucopolysaccharidosis type IIIA (MPS IIIA). In some embodiments, the selective or specific agonist of the CB2 cannabinoid receptor is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor selected from HU-308, JWH-015, GW-405833 (L-798872), AM-1871, JWH-133, GW-842166X, and a CB2 agonist compound disclosed herein. In some embodiments, the method treats the lysosomal storage disease or disorder or results in the improvement of one or more symptoms associated with the lysosomal storage disease or disorder. In some embodiments, the method prevents neuronal death associated with the lysosomal storage disease or disorder. In some embodiments, the method improves the subject’s cognition. In some embodiments, the method improves the subject’s learning and/or memory. In some embodiments, the method reduces inflammation levels in the subject. In some embodiments, the method reduces inflammation levels in the brain or CNS of the subject. In some embodiments, the method activates CB2 on microglia in the brain of the subject. In some embodiments, the method treats the reticuloendothelial system. In some embodiments, the method treats the skeletal system. In some embodiments, the method results in reduced inflammation levels in an organ other than the brain. In some embodiments, the organ other than the brain is selected from the liver, spleen, lungs, adrenal gland, kidney, heart, articular cartilage, articular joint space, and bone marrow. In some embodiments, the lifespan of the subject is increased as compared to the expected lifespan of an individual with the same lysosomal storage disease or disorder that has not been administered the selective agonist of the CB2 cannabinoid receptor. In some embodiments, the method further comprises administering one or more additional therapeutic agents. In some embodiments, the additional therapeutic agent comprises an enzyme replacement therapy, gene therapy, substrate reduction therapy, chaperone therapy, or a small molecule therapy. In some embodiments, the method further comprises administering a direct or indirect agonist of the CB1 cannabinoid receptor.

In some embodiments, the present disclosure includes a composition comprising an agent capable of selectively activating the CB2 cannabinoid receptor, for use in a method of treating a lysosomal disease or disorder. In some embodiments, the composition further comprises an agent capable of directly or indirectly activating the CB1 cannabinoid receptor.

In some embodiments, the present disclosure includes a pharmaceutical composition comprising an agent capable of selectively activating the CB2 cannabinoid receptor, for use in a method of treating a lysosomal disease or disorder and and one or more pharmaceutically acceptable salts, excipients or vehicles. In some embodiments, the pharmaceutical composition further comprises an agent capable of directly or indirectly activating the CB1 cannabinoid receptor. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

In some embodiments, the present disclosure includes a method of reducing beta amyloid and or tau protein levels in the brain of subject with a lysosomal storage disease or disorder comprising administering to the subject a selective or specific agonist of the CB2 cannabinoid receptor. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the ttransport diseases is selected from Cystinosis and Salla Disease. In some embodiments, the neuronal ceroid lipofuscinosis is Batten’s disease. In some embodiments, the lysosomal storage disease or disorder is Farber Diseaseor mucopolysaccharidosis type IIIA (MPS IIIA). In some embodiments, the selective or specific agonist of the CB2 cannabinoid receptor is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor selected from HU-308, JWH-015, GW-405833 (L-7681212), AM-11211, JWH-133, GW-842166X, and a CB2 agonist compound disclosed herein. In some embodiments, the method treats the lysosomal storage disease or disorder or results in the improvement of one or more symptoms associated with the lysosomal storage disease or disorder. In some embodiments, the method prevents neuronal death associated with the lysosomal storage disease or disorder. In some embodiments, the method improves the subject’s cognition. In some embodiments, the method improves the subject’s learning and/or memory. In some embodiments, the method reduces inflammation levels in the subject. In some embodiments, the method activates CB2 on microglia in the brain of the subject. In some embodiments, the method reduces activation of microglia in the brain of the subject. In some embodiments, the method treats the reticuloendothelial system. In some embodiments, the method treats the skeletal system. In some embodiments, the method results in reduced inflammation levels in an organ other than the brain. In some embodiments, the organ other than the brain is selected from the liver, spleen, lungs, adrenal gland, kidney, heart, articular cartilage, articular joint space, and bone marrow. In some embodiments, the lifespan of the subject is increased as compared to the expected lifespan of an individual with the same lysosomal storage disease or disorder that has not been administered the selective agonist of the CB2 cannabinoid receptor. In some embodiments, the method further comprises administering one or more additional therapeutic agents. In some embodiments, the additional therapeutic agent comprises an enzyme replacement therapy, gene therapy, substrate reduction therapy, chaperone therapy, or a small molecule therapy. In some embodiments, the method further comprises administering a direct or indirect agonist of the CB1 cannabinoid receptor. In some embodiments, the CB2 agonist results in an improvement in the subject’s myelination. In some embodiments, the CB2 agonist inhibits or reduces demyelination in the subject. In some embodiments, the CB2 agonist inhibits or reduces astrocyte activation in the subject.

In some embodiments, the present disclosure includes a method of evaluating efficacy of a therapy for treating a lysosomal storage disease or disorder in a subject who previously received the therapy, comprising: a) obtaining a post-therapy expression level of CB2 cannabinoid receptor in the subject; and b) comparing the post-therapy expression level to a pre-therapy expression level of CB2 cannabinoid receptor in the subject, wherein a reduced expression level indicates an effective therapy and an elevated expression level indicates an ineffective therapy. In some embodiments, the therapy is an enzyme replacement therapy, gene therapy, bone-marrow transplantation, substrate reduction or inhibition therapy, glucocorticoid, inhibitor of histone deacetylase, chaperone therapy, or any combination thereof. In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. In some embodiments, the lipidoses is selected Gaucher disease, Fabry disease, Farber Disease, Krabbe Disease, Metachromatic Leukodystrophy, Acid Spingomyelinase Deficiency (ASMD), Niemann-Pick Disease Type C, Gangliosidoses 1, and Gangliosidoses 2. In some embodiments, the mucopolysaccharidoses is selected from MPS types I, II, III, IV, VI, VII and IX. In some embodiments, the Mucolipidoses is selected from Mucolipidoses I, Mucolipidoses 2, Mucolipidoses 3 and Mucolipidoses 4. In some embodiments, the glycogen storage disease is Pompe Disease. In some embodiments, the carbohydrate storage diseases is selected from Mannosidosis and Sialidosis. In some embodiments, the transport diseases is selected from Cystinosis and Salla Disease. In some embodiments, the neuronal ceroid lipofuscinosis is Batten’s disease. In some embodiments, the method further comprises administering to the subject a selective or specific agonist of the CB2 cannabinoid receptor. In some embodiments, the selective or specific CB2 agonist is administered to the subject as a pharmaceutical composition. In some embodiments, the pharmaceutical composition comprises the selective or specific agonist of the CB2 cannabinoid receptor and one or more pharmaceutically acceptable salts, excipients or vehicles. In some embodiments, the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants. In some embodiments, the selective or specific agonist of the CB2 cannabinoid receptor selected from HU-308, JWH-015, GW-405833 (L-768242), AM-1241, JWH-133, GW-842166X, and a CB2 agonist compound disclosed herein. In some embodiments, the method prevents neuronal death associated with the lysosomal storage disease or disorder. In some embodiments, the method improves the subject’s cognition, learning and/or memory.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows CB2 cannabinoid receptor expression in the liver of 10 month-old MPS IIIA mice. FIG. 1A shows CB2 mRNA levels in the liver of the MPS IIIA mice relative to wild type (wt). FIG. 1B shows representative CB2 protein staining in the liver of wt and MPS IIIA mice. Topro in blue shows cell nuclei.

FIG. 2 shows representative images of CB2 cannabinoid receptor protein staining in the cortex of wt and MPS IIIA mice. Topro in blue shows cell nuclei.

FIG. 3 shows representative images of CB2 cannabinoid receptor protein staining in 8.5 week-old wt and Farber mice. For all FIG. 3 panels, Topro in blue shows cell nuclei. FIG. 3A shows staining in the liver in wt (top row) and Farber mice (bottom row), with 10X, 20, and 40X magnification shown as indicated. FIG. 3B shows staining in the spleen in wild type (top row) and Farber mice (bottom row), with 10X, 20, and 40X magnification shown as indicated. FIG. 3C shows staining in the cerebellum in wild type (top left and top middle panels) and Farber mice (bottom left and bottom middle panels), and the cortex of wt (top right panel) and Farber mice (bottom right panel).

FIG. 4 shows body weight (y axis) and life span (x axis) of Farber mice treated with vehicle control (DMSO) or with JWH133, a CB2 selective agonist. On day 58 all JWH133-treated mice were sacrificed for biochemical and histological analyses. All of the vehicle control mice had already died at this point, and there was a significant prevention of weight loss.

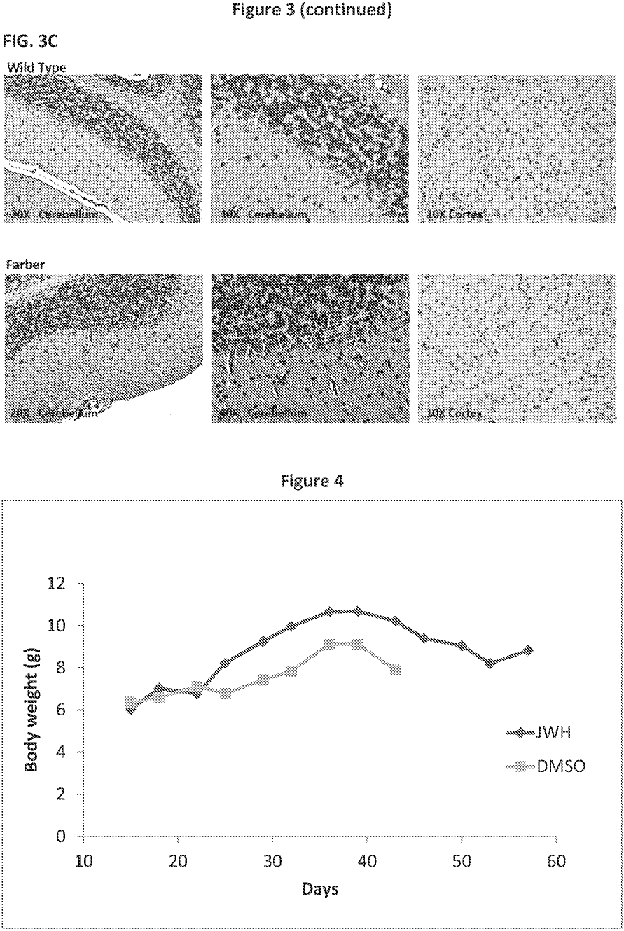

FIG. 5 shows representative images of CB2 cannabinoid receptor protein staining in 8.5 week-old Farber mice treated by enzyme replacement therapy (bottom row) compared to untreated Farber mice (top row), with 10X, 20, and 40X magnification shown as indicated.

FIG. 6 shows representative images of CB2 cannabinoid receptor protein staining in 8.5 week-old Farber mice treated by CB2 agonist JWH133 (bottom row) compared to untreated Farber mice (top row), with 10X, 20, and 40X magnification shown as indicated.

FIG. 7 shows representative images of immunohistochemical analysis of MCP-1 expression in liver sample of 8.5 week-old Farber mice treated by CB2 agonist JWH133 (bottom row) compared to untreated Farber mice (top row), with 10X, 20, and 40X magnification shown as indicated.

FIG. 8 shows representative result of MCP-1 expression in plasma of 8.5 week-old Farber mice treated by CB2 agonist JWH133 compared to untreated Farber mice.

FIG. 9 shows representative result of MCP-1 level released by fibroblasts from MPS IIIA treated by CB2 agonist JWH133 or PM226 compared to untreated cells.

### DETAILED DESCRIPTION OF THE INVENTION

The practice of the present invention will employ, unless indicated specifically to the contrary, conventional methods of molecular biology, recombinant DNA techniques, protein expression, and protein / peptide / carbohydrate chemistry within the skill of the art, many of which are described below for the purpose of illustration. Such techniques are explained fully in the literature. See, e.g., Sambrook, et al., Molecular Cloning: A Laboratory Manual (3^{rd} Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R.I. (2005) Culture of Animal Cells, a Manual of Basic Technique, 5^{th} Ed. Hoboken NJ, John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005). Poly(ethylene glycol), Chemistry and Biological Applications, ACS, Washington, 1997; Veronese, F., and J.M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### DEFINITIONS AND ABBREVIATIONS

Unless defined otherwise, all technical and scientific terms used herein have the same meaning as commonly understood by one of ordinary skill in the art to which this invention belongs. As used in the specification and appended claims, unless specified to the contrary, the following terms have the meaning indicated. With regard to this specification, any time a definition of a term as defined herein, differs from a definition given for that same term in an incorporated reference, the definition explicitly defined herein is the correct definition of the term.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

A “composition” can comprise an active agent and a carrier, inert or active, e.g., a pharmaceutically acceptable carrier, diluent or excipient. In particular embodiments, the compositions are sterile, substantially free of endotoxins or non-toxic to recipients at the dosage or concentration employed.

Unless the context requires otherwise, throughout the present specification and claims, the word “comprise” and variations thereof, such as, “comprises” and “comprising” are to be construed in an open and inclusive sense, that is, as “including, but not limited to”.

By “consisting of” is meant including, and limited to, whatever follows the phrase “consisting of.” Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “biological activity” or “bioactivity” refers to any response induced in an in vitro assay or in a cell, tissue, organ, or organism, (e.g., an animal, or a mammal, or a human) as the result of administering any compound, agent, polypeptide, conjugate, pharmaceutical composition contemplated herein. Biological activity may refer to agonistic actions or antagonistic actions. The biological activity may be a beneficial effect; or the biological activity may not be beneficial, i.e. a toxicity. In some embodiments, biological activity will refer to the positive or negative effects that a drug or pharmaceutical composition has on a living subject, e.g., a mammal such as a human. Accordingly, the term “biologically active” is meant to describe any compound possessing biological activity, as herein described. Biological activity may be assessed by any appropriate means currently known to the skilled artisan. Such assays may be qualitative or quantitative. The skilled artisan will readily appreciate the need to employ different assays to assess the activity of different polypeptides; a task that is routine for the average researcher. Such assays are often easily implemented in a laboratory setting with little optimization requirements, and more often than not, commercial kits are available that provide simple, reliable, and reproducible readouts of biological activity for a wide range of polypeptides using various technologies common to most labs. When no such kits are available, ordinarily skilled researchers can easily design and optimize in-house bioactivity assays for target polypeptides without undue experimentation; as this is a routine aspect of the scientific process.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

Reference throughout this specification to “embodiment” or “one embodiment” or “an embodiment” or “some embodiments” or “certain embodiments” means that a particular feature, structure or characteristic described in connection with the embodiment is included in at least one embodiment of the present invention. Thus, the appearances of the phrases “in one embodiment” or “in an embodiment” or “in certain embodiments” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. Similarly, a “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The terms “in vitro”, “ex vivo”, and “in vivo” are intended herein to have their normal scientific meanings. Accordingly, e.g., “in vitro” is meant to refer to experiments or reactions that occur with isolated cellular components, such as, e.g., an enzymatic reaction performed in a test tube using an appropriate substrate, enzyme, donor, and optionally buffers / cofactors. “Ex vivo” is meant to refer to experiments or reactions carried out using functional organs or cells that have been removed from or propagated independently of an organism. “In vivo” is meant to refer to experiments or reactions that occur within a living organism in its normal intact state.

“Mammal” includes humans and both domestic animals such as laboratory animals and household pets, (e.g., cats, dogs, swine, cattle, sheep, goats, horses, and rabbits), and non-domestic animals such as wildlife and the like.

“Optional” or “optionally” means that the subsequently described event, or circumstances, may or may not occur, and that the description includes instances where said event or circumstance occurs and instances in which it does not.

“Pharmaceutical composition” refers to a formulation of a compound (e.g. a therapeutically useful polypeptide) and a medium generally accepted in the art for the delivery of the compound to an animal, e.g., humans. Such a medium may include any pharmaceutically acceptable carriers, diluents or excipients therefore.

“Pharmaceutically effective excipients” and “pharmaceutically effective carriers” are well known to those of skill in the art, and methods for their preparation are also readily apparent to the skilled artisan. Such compositions, and methods for their preparation, may be found, e.g., in Remington’s Pharmaceutical Sciences, 19th Edition (Mack Publishing Company, 1995, incorporated herein).

The terms “polynucleotide”, “nucleotide”, “nucleotide sequence”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may include non-nucleotide components. A polynucleotide may be further modified after polymerization, such as by conjugation with a labeling component.

A “subject,” as used herein, includes any animal that exhibits a disease or symptom, or is at risk for exhibiting a disease or symptom, which can be treated with an agent of the invention. Suitable subjects include laboratory animals (such as mouse, rat, rabbit, or guinea pig), farm animals, and domestic animals or pets (such as a cat or dog). Non-human primates and, preferably, human patients, are included.

“Substantially” or “essentially” means of ample or considerable amount, quantity, size; nearly totally or completely; for instance, 95% or greater of some given quantity.

“Therapeutic agent” refers to any compound that, when administered to a subject, (e.g., preferably a mammal, more preferably a human), in a therapeutically effective amount is capable of effecting treatment of a disease or condition as defined below.

“Therapeutically effective amount” or “Therapeutically effective dose” refers to an amount of a compound of the invention that, when administered to a subject, (e.g., preferably a mammal, more preferably a human), is sufficient to effect treatment, as defined below, of a disease or condition in the animal. The amount of a compound of the invention that constitutes a “therapeutically effective amount” will vary depending on the compound, the condition and its severity, the manner of administration, and the age of the animal to be treated, but can be determined routinely by one of ordinary skill in the art having regard to his own knowledge and to this disclosure.

“Treating” or “treatment” as used herein covers the treatment of the disease or condition of interest in a subject, preferably a human, having the disease or condition of interest, and includes: (i) preventing or inhibiting the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady, injury or condition may not have a known causative agent (so that etiology has not yet been worked out), and it is, therefore, not yet recognized as an injury or disease but only as an undesirable condition or syndrome, wherein a more or less specific set of symptoms have been identified by clinicians.

### OVERVIEW

Embodiments of the present disclosure include methods for treating or preventing lysosomal storage diseases and disorders in a subject in need thereof, comprising activating the CB2 cannabinoid receptor.

CB2 receptors are membrane bound, G-protein receptors that are a key component of the endocannabinoid system (ECS). The ECS consists of endogenous cannabinoids (endocannabinoids), the cannabinoid receptors CB1 and CB2, and various enzymes responsible for synthesizing and degrading endocannabinoids. Although widely distributed, CB2 receptors are primarily expressed on the surface of immune cells, including peripheral macrophages and microglia of the brain. In contrast, CB1 recptors are primarily found on neurons, and activation of this receptor is associated with psychotropic effects. Numerous signaling functions have been attributed to CB2 receptors including reduced inflammation and reduced cell death. As such, numerous small molecules have been developed to activate this receptor and many have been evaluated in animal models of inflammatory and other diseases.

The present disclosure is based in part on the surprising discovery that the expression of the cannabinoid receptor CB2 is significantly increased in the CNS and periphery of subjects having the lysosomal storage diseases MPS IIIA and Farber disease, which have diverse molecular pathogenesis, but each exhibit aberrant substrate storage (e.g., GAGs and lipids, respectively), and resultant inflammation and neurodegeneration. Further, when compositions comprising CB2 agonists are administered in vivo, to mice having Farber disease, increased body weight and increased lifespan results.

Accordingly, some embodiments of the present disclosure include methods of treating or preventing a lysosomal storage disease or disorder in a subject in need thereof, comprising administering to the subject one or more agonists of the CB2 cannabinoid receptor. In particular embodiments, the agonist of the CB2 receptor is a selective or specific agonists of the CB2 cannabinoid receptor, thereby minimizing the psychotropic effects caused by CB1 agonism. However, as is discussed below, in certain embodiments, particularly those with sphingomyelin storage, it is useful to activate the CB2 receptor with a compound that acts on both the CB1 and CB2 receptors (e.g., in the treatment of lipid storage disorders such as Acid Sphingomyelinase Deficiency (ASMD), Type A Niemann Pick disease (NPA), Type B Niemann Pick disease (NPB), Type C Niemann Pick disease (NPC)). In some embodiments, the term “selective agonist” of the CB2 cannabinoid receptor is used herein to refer to a compound that selectively activates the CB2 cannabinoid receptor over the CB1 cannabinoid receptor. In some embodiments, the term “specific agonist” of the CB2 cannabinoid receptor is used herein to refer to a compound that specifically activates the CB2 cannabinoid receptor and does not detectably activate the CB1 cannabinoid receptor. Accordingly, reference herein to “specific or selective agonists” (or “selective or specific agonists”) refers to such selective agonists or specific agonists of the CB2 cannabinoid receptor.

In some embodiments, the present disclosure provides a method of treating or preventing inflammation in a subject that has a lysosomal storage disease or disorder, comprising administering to the subject one or more agonist of the CB2 cannabinoid receptor In particular embodiments, the agonist of the CB2 receptor is a selective or specific agonists of the CB2 cannabinoid receptor, thereby minimizing the psychotropic effects caused by CB1 agonism.

In some embodiments, the present disclosure provides a method of inhibiting a loss of motor skills, cognitive decline, and/or systemic organ pathology associated with a lysosomal storage disease or disorder in a subject in need thereof, comprising administering to the subject one or more agonist of the CB2 cannabinoid receptor. In particular embodiments, the agonist of the CB2 receptor is a selective or specific agonists of the CB2 cannabinoid receptor, thereby minimizing the psychotropic effects caused by CB1 agonism.

In some embodiments, the present disclosure provides a method of inhibiting macrophage activation in a subject with a lysosomal storage disease or disorder comprising administering to the subject one or more agonist of the CB2 cannabinoid receptor. In particular embodiments, the agonist of the CB2 receptor is a selective or specific agonists of the CB2 cannabinoid receptor, thereby minimizing the psychotropic effects caused by CB1 agonism.

In some embodiments, the present disclosure provides a method of inhibiting astrocyte activation in a subject with a lysosomal storage disease or disorder comprising administering to the subject one or more agonist of the CB2 cannabinoid receptor. In particular embodiments, the agonist of the CB2 receptor is a selective or specific agonists of the CB2 cannabinoid receptor, thereby minimizing the psychotropic effects caused by CB1 agonism.

In some embodiments, the present disclosure provides a method of reducing at least one inflammatory cytokine or chemokine in a subject with a lysosomal storage disease or disorder comprising activating the CB2 receptor. In particular embodiments, the CB2 receptor is activated by administering to the subject one or more agonist of the CB2 receptor. In certain embodiments, the agonist of the CB2 receptor is a selective or specific agonist of the CB2 receptor, thereby minimizing the psychotropic effects caused by CB1 agonism.

In some embodiments, the present disclosure provides a method of reducing beta amyloid and or tau protein levels in the brain of subject with a lysosomal storage disease or disorder comprising administering to the subject one or more agonist of the CB2 cannabinoid receptor. In particular embodiments, the agonist of the CB2 receptor is a selective or specific agonists of the CB2 cannabinoid receptor, thereby minimizing the psychotropic effects caused by CB1 agonism.

In some embodiments, the present disclosure provides a method of inhibiting demyelination associated with a lysosomal storage disease or disorder in a subject in need thereof, comprising administering to the subject one or more agonist of the CB2 cannabinoid receptor. In particular embodiments, the agonist of the CB2 receptor is a selective or specific agonists of the CB2 cannabinoid receptor, thereby minimizing the psychotropic effects caused by CB1 agonism.

In some embodiments, the present disclosure includes methods of treating a lysosomal storage disease or disorder in a subject that has high expression of CB2, the method comprising administering to the subject a CB2 agonist. The CB2 agonist may be a selective or specific agonist of the CB2 receptor. The high expression of CB2 may be in the periphery. The high expression of CB2 may be in the CNS. The high expression of CB2 may be in both the CNS and the periphery. The expression levels of CB2 in the periphery may be used as a corollary for the expression levels of CB2 in the CNS in some embodiments. In some embodiments, the administering of the CB2 agonist to the subject may occur before it has been determined that the subject has high expression of CB2. Alternatively, in particular embodiments, the administering of the CB2 agonist to the subject occurs after it has been determined that the subject has high expression of CB2. In some embodiments, the method comprises administering the CB2 agonist (e.g., a selective or specific agonist of the CB2 receptor) to the subject, wherein it has been indirectly determined that the subject has high CNS expression of the CB2 receptor based on the expression levels of CB2 in the periphery of the subject. For example, in one embodiment, it is shown that a patient has high expression of CB2 on circulating macrophages and this high expression is utilized as a corollary for the expression levels of CB2 in the subject’s CNS. In such an embodiment, certain subjects having a lysosomal storage disease or disorder and exhibiting high levels of CB2 expression in the periphery will be ideal candidates for treatment with a CB2 agonist. Thus, subjects having lysosomal storage diseases or disorders may be stratified based on their CB2 expression levels for treatment with a CB2 agonist in some embodiments.

Any agonist of the CB2 receptor known in the art or disclosed herein may be used in any one of the compositions or methods disclosed herein. As noted above, in particular embodiments, the agonist of the CB2 receptor is a selective or specific agonist of the CB2 cannabinoid receptor. Any selective or specific agonist of the CB2 receptor that is disclosed herein or known in the art may be used in the compositions and methods disclosed herein. For example, in some instances the selective or specific agonist of the CB2 cannabinoid receptor is selected from HU-308 (CAS No.: 256934-39-1), JWH-015 (CAS No.: 155471-08-2), GW-405,833 (L-768242) (CAS No.: 180002-83-9), AM-1241 (CAS No.: 444912-48-5), JWH-133 (CAS No.: 259869-55-1), GW-842166 (CAS No.: 666260-75-9), S-777469 (CAS No.: 885496-53-7), JBT-101 (CAS No.: 137945-48-3), JWH-018 (CAS No.: 209414-07-3); WIN 55,212-2 (CAS No.: 131543-22-1); HU-210 (CAS No.: 112830-95-2), or a combination thereof. In some instances, the selective or specific agonist of the CB2 cannabinoid receptor is selected from the following compounds:

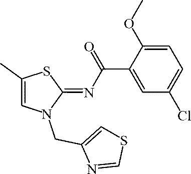 [SMILES: COc1ccc(Cl)cc1C(=O)/N=c1\sc(C)cn1Cc1cscn1]

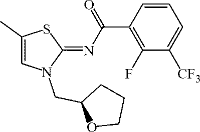 [SMILES: Cc1cn(C[C@H]2CCCO2)/c(=N/C(=O)c2cccc(C(F)(F)F)c2F)s1]

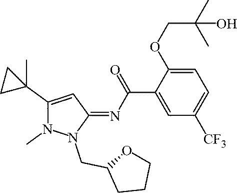

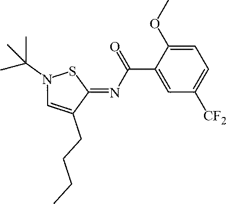

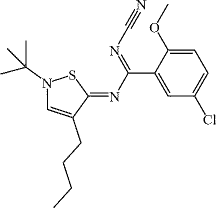 [SMILES: CCCCc1cn(C(C)(C)C)s/c1=N\C(=N\C#N)c1cc(Cl)ccc1OC]

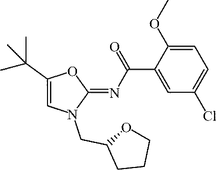

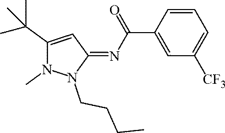 [SMILES: CCCCn1/c(=N/C(=O)c2cccc(C(F)(F)F)c2)cc(C(C)(C)C)n1C]

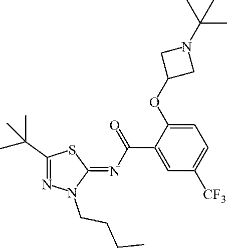 [SMILES: CCCCn1nc(C(C)(C)C)s/c1=N\C(=O)c1cc(C(F)(F)F)ccc1OC1CN(C(C)(C)C)C1]

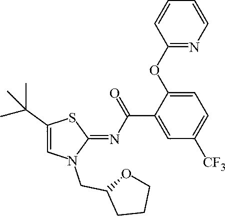

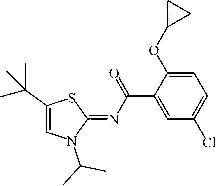 [SMILES: CC(C)n1cc(C(C)(C)C)s/c1=N\C(=O)c1cc(Cl)ccc1OC1CC1]

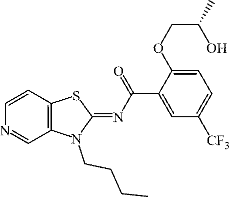 [SMILES: CCCCn1/c(=N/C(=O)c2cc(C(F)(F)F)ccc2OC[C@H](C)O)sc2ccncc21]

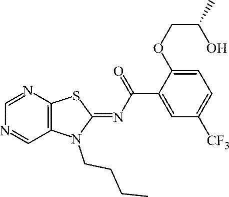 [SMILES: CCCCn1/c(=N/C(=O)c2cc(C(F)(F)F)ccc2OC[C@H](C)O)sc2ncncc21]

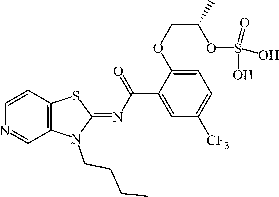

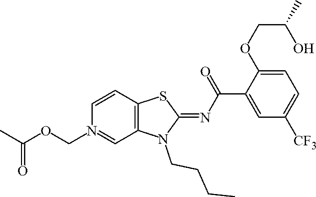

In some embodiments, the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis. For example, in some instances the lipidoses is Gaucher disease, Fabry disease, Farber Disease, Krabbe Disease, Metachromatic Leukodystrophy, Niemann-Pick Disease, Gangliosidoses 1, or Gangliosidoses 2. In some instances, the mucopolysaccharidoses is MPS type I, MPS type II, MPS type III, MPS type IV, MPS type VI, MPS type VII, or MPS type IX. In some instances, the Mucolipidoses is Mucolipidoses I, Mucolipidoses 2, Mucolipidoses 3 or Mucolipidoses 4. In some instances, the glycogen storage disease is Pompe Disease. In some instances, the carbohydrate storage disease is Mannosidosis or Sialidosis. In some instances, the transport disease is Cystinosis or Salla Disease. In some instances, the neuronal ceroid lipofuscinosis is Batten’s disease. In some embodiments, the lysosomal storage disease or disorder is selected from the group consisting of Niemann-Pick Type A and Niemann-Pick Type B, also known as Acid Sphingomyelinase Deficiency (ASMD), Niemann-Pick Type C (NPA, NPB, or NPC respectively) or Mucopolysaccharidosis type IIIA (MPS IIIA).

In some embodiments, any one of the methods disclosed herein may optionally also comprise administering to the subject an additional therapeutic agent in combination with the agonist of CB2. Such combination therapies may include, e.g., any additional therapeutic agent known or expected to be useful in treating one or more symptom or underlying cause of the lysosomal storage disease or disorder. The combination of the agonist of CB2 and the additional agent may result in additive treatment of the lysosomal storage disease or disorder. The combination of the agonist of CB2 and the additional agent may result in synergistic treatment of the lysosomal storage disease or disorder.

Suitable combinations include, without limitation an agonist of the CB2 receptor (e.g., a selective agonist of the CB2 receptor or a specific agonist of the CB2 receptor) and an additional agent or therapy selected from an enzyme replacement therapy (including, e.g., a recombinant enzyme); a gene therapy (including e.g., a specific DNA sequence), substrate reduction or inhibition therapy; a glucocorticoid; an inhibitor of histone deacetylase; and a chaperone therapy. Suitable combinations include, without limitation an agonist of the CB2 receptor (e.g., a selective agonist of the CB2 receptor or a specific agonist of the CB2 receptor) and an HSP70 inhibitor (e.g., arimoclomol). Suitable combinations include, without limitation an agonist of the CB2 receptor (e.g., a selective agonist of the CB2 receptor or a specific agonist of the CB2 receptor) and an inhibitor of ceramide production. Suitable combinations include, without limitation an agonist of the CB2 receptor (e.g., a selective agonist of the CB2 receptor or a specific agonist of the CB2 receptor) and an anti-inflammatory therapy (e.g., for example PPS).

In some aspects, the additional agent that is administered to the subject in combination with the agonist of the CB2 receptor (e.g., a selective or specific agonist of the CB2 receptor) may be, e.g., an enzyme replacement therapy. The enzyme replacement therapy that may be used in combination with the agonist of the CB2 receptor may comprise administration of a recombinant enzyme to the subject. The enzyme may be delivered in any suitable form. For example, in some non-limiting aspects, the enzyme is delivered to the subject in the form of a purified protein. Thus, the enzyme replacement therapy may comprise, e.g., administration of IV infused recombinant enzyme, administration of intrathecally delivered recombinant enzyme, administration of intraventricular delivered recombinant enzyme, etc., wherein the recombinant enzyme may in some aspects be purified. In some aspects, such recombinant enzyme may be delivered to the subject as a composition. The composition may be a pharmaceutically acceptable composition. Such pharmaceutically acceptable compositions are known in the art and are disclosed herein. In some aspects, the composition comprising the recombinant enzyme is substantially free of endotoxin. In some aspects, the composition comprising the recombinant enzyme further comprises one or more carrier, excipient, diluent, and/or surfactant. The recombinant enzyme and the agonist of the CB2 receptor may be administered to the patient simultaneously or separately. The agonist of the CB2 receptor and the recombinant enzyme may be formulated together in a single pharmaceutical composition or they may be formulated separately in different formulations. When formulated separately, the agonist of the CB2 receptor may be administered before or after the recombinant enzyme.

In some non-limiting aspects, the delivery of the enzyme to the subject may be by administering an autologous cell expressing a gene encoding a functional enzyme to the subject. The autologous cell may be a hematopoietic stem cell. The autologous hematopoietic stem cell may be genetically engineered to express a gene encoding the functional enzyme. The gene encoding the functional enzyme may be introduced into the autologous cell via viral infection. Any suitable viral packaging may be utilized in delivering the enzyme to the desired tissue. For example, the viral infection may be mediated by a recombinant retrovirus, a recombinant lentivirus, or a recombinant adeno-associated virus (AAV). The AAV may be of any suitable serotype. Those of average skill in the art will appreciate that different serotypes of AAV vectors may be more suitable for delivery to different tissues. For example, AAV9 serotype AAV vectors may be suitable for delivery of an ASM enzyme by means of, a cerebellomedullary cistern injection. Other AAV serotypes may also be used.

In some non-limiting aspects, the delivery of the enzyme to the subject may be by administering a recombinant virus to the subject, wherein infection with the virus causes the infected cell to express a gene encoding a functional enzyme. The administering of the recombinant virus may be via any suitable means. For example, the administration of the virus may be by direct injection of the recombinant virus into subject. For instance, the virus may be injected into the subject’s brain. The virus may be injected into a ventricle of the subject. The virus may be administered to the subject via a route of administration selected from intravenous infusion, intrathecal infusion, and intraventricular injection.

In some aspects, the additional agent that is administered to the subject in combination with the agonist of the CB2 receptor (e.g., a selective or specific agonist of the CB2 receptor) may be, e.g., a glucocorticoid. The glucocorticoid may be dexamethasone. The glucocorticoid may be hydrocortisone.

The glucocorticoid may be delivered in any suitable form. For example, the glucocorticoid may be delivered to the subject as a composition. The composition may be a pharmaceutically acceptable composition. Such pharmaceutically acceptable compositions are known in the art and are disclosed herein. In some aspects, the composition comprising the glucocorticoid is substantially free of endotoxin. In some aspects, the composition comprising the glucocorticoid further comprises one or more carrier, excipient, diluent, and/or surfactant. The glucocorticoid and the agonist of the CB2 receptor may be administered to the patient simultaneously or separately. The agonist of the CB2 receptor and the glucocorticoid may be formulated together in a single pharmaceutical composition or they may be formulated separately in different formulations. When formulated separately, the agonist of the CB2 receptor may be administered before or after glucocorticoid.

In some aspects, the additional agent that is administered to the subject in combination with the agonist of the CB2 receptor (e.g., a selective or specific agonist of the CB2 receptor) may be, e.g., an inhibitor of histone deacetylase. In some aspects, the additional agent that is administered to the subject in combination with the agonist of the CB2 receptor may be, e.g., an agent that enhances plasma membrane calcium ATPase (PMCA) expression. The additional agent that is administered to the subject in combination with the agonist of the CB2 receptor may be, e.g., the histone deacetylase inhibitor SAHA (Suberoylanilide hydroxamic acid). The histone deacetylase inhibitor (e.g., SAHA) may be delivered in any suitable form. For example, the histone deacetylase inhibitor (e.g., SAHA) may be delivered to the subject as a composition. The composition may be a pharmaceutically acceptable composition. Such pharmaceutically acceptable compositions are known in the art and are disclosed herein. In some aspects, the composition comprising the histone deacetylase inhibitor (e.g., SAHA) is substantially free of endotoxin. In some aspects, the composition comprising the histone deacetylase inhibitor (e.g., SAHA) further comprises one or more carrier, excipient, diluent, and/or surfactant. The histone deacetylase inhibitor (e.g., SAHA) and the agonist of the CB2 receptor may be administered to the patient simultaneously or separately. The agonist of the CB2 receptor and the histone deacetylase inhibitor (e.g., SAHA) may be formulated together in a single pharmaceutical composition or they may be formulated separately in different formulations. When formulated separately, The agonist of the CB2 receptor may be administered before or after the histone deacetylase inhibitor (e.g., SAHA).

The agonist of the CB2 receptor may be administered in combination with a chaperone or substrate reduction therapy. The chaperone therapy may be an inhibitor of α-GalA. The substrate therapy may be Miglustat. The agonist of the CB2 receptor may be administered in combination with a small molecule inhibitor that is useful in treating a lysosomal storage disorder. Thus, the agonist of the CB2 receptor may be administered, e.g., in combination with a substrate reduction therapy. The substrate reduction therapy may inhibit ganglioside production. The additional agent may be an inhibitor of sphingomyelin production. The additional agent may be an inhibitor of sphingomyelin synthase. The additional agent may be an inhibitor of glucosylceramide synthase. The additional agent may be a ceramide analogue. The additional agent may be Miglustat. The additional agent may be Eliglustat. The substrate reduction therapy may be Zavesca or Cerdelga.

The chaperone therapy may be delivered in any suitable form. For example, the chaperone therapy may be delivered to the subject as a composition. The composition may be a pharmaceutically acceptable composition. Such pharmaceutically acceptable compositions are known in the art and are disclosed herein. In some aspects, the composition comprising the chaperone therapy is substantially free of endotoxin. In some aspects, the composition comprising the chaperone therapy further comprises one or more carrier, excipient, diluent, and/or surfactant. The chaperone therapy and the agonist of the CB2 receptor may be administered to the patient simultaneously or separately. The agonist of the CB2 receptor and the chaperone therapy may be formulated together in a single pharmaceutical composition or they may be formulated separately in different formulations. When formulated separately, the agonist of the CB2 receptor may be administered before or after the chaperone therapy.

In certain embodiments, the agonist of the CB2 receptor may be administered in combination with an additional agent that induces reduction of high levels of sphingomyelin, sphingosine and / or gangliosides GM2 and GM3. The agonist of the CB2 receptor may be administered in combination with an inhibitor of ceramide. The agonist of the CB2 receptor may be administered in combination with an additional agent that induces reduction of high levels of cytosolic calcium in neurons. The agonist of the CB2 receptor may be administered in combination with an additional agent that induces reduction of oxidative stress. The agonist of the CB2 receptor may be administered in combination with an additional agent that induces enhancement of glucocorticoid signaling (e.g., an agent that enhances expression of glucocorticoid receptors or increases glucocorticoid levels). The agonist of the CB2 receptor may be administered in combination with an additional agent that induces reduction of inflammation with two main objectives: Reduction of pro-inflammatory microglia (M1 type) and/or the secretion of pro-inflammatory cytokines or chemokines or enhancement of phagocytic microglia (M2 type).

Moreover, in some embodiments, any one of the combination therapies disclosed herein (i.e., the agonist of the CB2 receptor and the additional agent) may comprise a second, third, fourth additional agent, or more than four additional agents. Optionally, the additional agent or agents may comprise a second agonist of the CB2 receptor inhibitor. Alternatively, the additional agent or agents may comprise an agent other than a second agonist of the CB2 receptor inhibitor.

In some aspects, the present disclosure provides a combination therapy for treating a lipid storage disorder in a subject in need thereof comprising administering to the subject an agonist of the CB2 receptor and an agonist of the CB1 receptor. In some embodiments, the lipid storage disorder is Acid Sphingomyelinase Deficiency (ASMD). In some embodiments the ASMD is NPA or NPB. In some embodiments, the lipid storage disorder is Niemann-Pick Type C. In some embodiments, the agonist of the CB2 receptor is a selective or specific agonist of the CB2 receptor. In some embodiments, the agonist of the CB1 receptor is an indirect CB1 agonist. For example, in some particular embodiments, the indirect agonist of the CB1 receptor is an agent that inhibits the natural degradation of an endogenous CB1 agonist thereby increasing the levels of the endogenous CB1 agonist leading to increased CB1 signaling. In particular embodiments, the indirect agonist of the CB1 receptor is a fatty acid amide hydrolase inhibitor (FAAHi). We have previously shown that such FAAHi’s are effective at reducing sphingomyelin and normalizing other lysosomal substrates by activating the CB1 receptor in subjects with lipid storage disorders (WO 2019/173394, incorporated herein by reference in its entirety). Any FAAHi known in the art or disclosed herein may be used in such a method. For example, in some particular embodiments, the FAAHi selected from PF-04457845 (CAS No.: 1020315-31-4), AM-374 (CAS No.: 86855-26-7), AM-3506, AM-5206, ARN2508, IPI-940, IW-6118, BIA-102474 (CAS No.: 1233855-46-3), BMS-469908, JNJ-1661010, JNJ-40413269, JNJ-42165279 (CAS No.: CAY-10402), PF-3845, PF-750, SSR411298, URB597 (CAS No.: 546141-08-6), URB694, URB937, SSR-101010, and V158866. In some embodiments, the FAAHi is administered to the subject as a pharmaceutical composition. In particular embodiments, the FAAHi is PF-04457845. The PF-04457845 may be administered to the subject as a pharmaceutical composition. In some embodiments, instead of using an agonist of the CB1 receptor and an agonist of the CB2 receptor, the method of treating the lipid storage disorder comprises administering to the subject in need thereof an agent that activates both the CB1 and the CB2 receptors (referred to herein as a “dual CB receptor agonist”). Any dual CB receptor agonist known in the art or disclosed herein may be used in such a method. In one particular embodiment, the dual CB receptor agonist is S-444823 (CAS No.: 885490-15-3). Such methods may also comprise administering to the subject another agent selected from an Enzyme Replacement Therapy (ERT), a gene therapy, a substrate reduction/substrate inhibition; a small molecule; a chaperone, and an enzyme activator. The additional agent may be any additional agent disclosed herein.

Administration of the disclosed compositions and compounds (e.g., an agonist of the CB2 receptor, agonist of the CB1 receptor, FAAHi, and/or other therapeutic agents) can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Pharmaceutical compositions suitable for the delivery of an agent such as the agonist of the CB2 receptor (alone or, e.g., in combination with another therapeutic agent according to the present disclosure) and methods for their preparation will be readily apparent to those skilled in the art. Such compositions and methods for their preparation may be found, e.g., in Remington’s Pharmaceutical Sciences, 19th Edition (Mack Publishing Company, 1995), incorporated herein in its entirety.

Illustrative pharmaceutical compositions are tablets and gelatin capsules comprising a agonist of the CB2 receptor alone or in combination with another therapeutic agent according to the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, an agonist of the CB2 receptor (alone or in combination with another therapeutic agent according to the disclosure) is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the agonist of the CB2 receptor (alone or in combination with another therapeutic agent according to the disclosure).

The agonist of the CB2 receptor (or an additional agent utilized in a combination therapy) can be also formulated as a suppository, alone or in combination with another therapeutic agent according to the disclosure, which can be prepared from fatty emulsions or suspensions; using polyalkylene glycols such as propylene glycol, as the carrier.

The agonist of the CB2 receptor (or an additional agent utilized in a combination therapy) can also be administered in the form of liposome delivery systems, such as small unilamellar vesicles, large unilamellar vesicles and multilamellar vesicles, either alone or in combination with another therapeutic agent according to the disclosure. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

The one or more agonist of the CB2 receptor can also be delivered by the use of monoclonal antibodies as individual carriers to which the disclosed compounds are coupled. An agonist of the CB2 receptor can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a FAAHi inhibitor can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising an agonist of the CB2 receptor (alone or in combination with another therapeutic agent according to the present disclosure) and a pharmaceutically acceptable carrier. The pharmaceutically acceptable carrier can further include an excipient, diluent, or surfactant.

The agonist of the CB2 receptor utilized in the compositions and methods disclosed herein need not be a single agonist of the CB2 receptor (although as is clearly set forth above, the agonist of the CB2 receptor may be a single agonist of the CB2 receptor). Instead, compositions and methods may comprise one or more agonist of the CB2 receptor and, optionally, an additional agent for use in a combination therapy disclosed herein.

Thus, the present disclosure provides in some embodiments, compositions (e.g., pharmaceutical compositions) comprising one or more agonist of the CB2 receptor for use in a method disclosed herein, e.g., an agonist of the CB2 receptor monotherapy or an agonist of the CB2 receptor combination therapy. Such compositions may comprise an agonist of the CB2 receptor and, e.g., one or more carrier, excipient, diluent, and/or surfactant.

The present disclosure provides, in some embodiments, compositions (e.g., pharmaceutical compositions) comprising one or more CB1 activator for use in a method disclosed herein, e.g., a CB1 agonist monotherapy or a CB1 agonist combination therapy. Such compositions may comprise a CB1 agonist and, e.g., one or more carrier, excipient, diluent, and/or surfactant. Such a CB1 agonist may be a direct agonist of an indirect agonist (e.g., a FAAHi).

The present disclosure provides, in some embodiments, a method of evaluating efficacy of a therapy for treating a lysosomal storage disease or disorder in a subject who previously received the therapy, comprising: a) obtaining a post-therapy expression level of CB2 cannabinoid receptor in the subject; and b) comparing the post-therapy expression level to a pre-therapy expression level of CB2 cannabinoid receptor in the subject, wherein a reduced expression level indicates an effective therapy and an elevated expression level indicates an ineffective therapy. In some embodiments, the therapy is an enzyme replacement therapy, gene therapy, bone-marrow transplantation, substrate reduction or inhibition therapy, glucocorticoid, inhibitor of histone deacetylase, chaperone therapy, or any combination thereof.

In order to fully illustrate the present invention and advantages thereof, the following specific examples are given, it being understood that the same are intended only as illustrative and in no way limitative.

### EXAMPLES

### Example 1

### Alterations of the Endocannabinoid Receptor CB2 in the Liver and Brain of MPSIIIA Mice, a Model for Sanfilippo Syndrome

### Background

Sanfilippo syndrome, or mucopolysaccharidosis III (MPS III), is a rare genetic disorder characterized by the deficiency of enzymes involved in the degradation of glycosaminoglycans (GAGs). In the absence of enzyme, partially degraded GAG molecules cannot be cleared from the body and accumulate in lysosomes of various tissues, resulting in progressive widespread somatic dysfunction (Neufeld and Muenzer, 2001).

Four distinct forms of MPS III, designated MPS III A, B, C, and D, have been identified. Each represents a deficiency in one of four enzymes involved in the degradation of the GAG heparan sulfate. All forms include varying degrees of the same clinical symptoms, including coarse facial features, hepatosplenomegaly, corneal clouding and skeletal deformities. Most notably, however, is the severe and progressive loss of cognitive ability, which is tied not only to the accumulation of heparan sulfate in neurons, but also the subsequent elevation of the gangliosides GM2, GM3 and GD2 caused by primary GAG accumulation (Walkley 1998).

Mucopolysaccharidosis type IIIA (MPS IIIA; Sanfilippo Syndrome Type A) is the most severe form of Sanfilippo syndrome and affects approximately 1 in 100,000 people worldwide. Sanfilippo Syndrome Type A (SanA) is characterized by a deficiency of the enzyme N-Sulfoglucosamine Sulfohydrolase, also known as heparin sulfamidase, which is involved in the lysosomal degradation of the mucopolysaccharide haparan sulphate. Mutations in the SGSH gene encoding heparan sulfamidase lead to heparan sulphate accumulation in neurons and glial cells, and to a lesser extend outside of the brain. A defining clinical feature of this disorder is central nervous system (CNS) degeneration, which results in loss of, or failure to attain, major developmental milestones. The progressive cognitive decline culminates in dementia and premature mortality. The disease typically manifests itself in young children, and the lifespan of an affected individual generally does not extend beyond late teens to early twenties.

The MPSIIIA mouse is a well-established natural model for SanA. These mice exhibit symptoms that reflect the human disease pathology and life span, displaying primarily CNS pathology with global accumulation of heparan sulphate glycosaminoglycans in somatic tissues.

The present study analyzed CB2 cannabinoid receptor expression in the liver and brain of MPSIIIA mice. The MPSIIIA mice we utilized were in the C57BL/6 strain, and in all experiments, mice were fed regular diet and provided water ad libitum from lixits. Samples of water were routinely analyzed for specified microorganisms and environmental contaminants. Environmental controls for the animal room were maintained at 22 ± 2° C., a relative humidity of 55 ± 10%, a minimum of 10-12 air changes/hour, and a 12-hour light/12-hour dark cycle.

### Methods

We analyzed mRNA and protein expression of the CB2 cannabinoid receptor in the liver of 10 month-old MPSIIIA mice via qPCR and immunofluorescence, respectively.

Briefly, mice were sacrificed and, following termination, they were transcardially perfused with phosphate-buffered saline (PBS). Brains were harvested and divided sagittaly in two hemispheres. Right hemisphere was blocked coronally in 2 mm blocks, snaps frozen by immersion in ice-cold isopentane and stored at -80° C. for posterior biochemical analysis. Left hemisphere was post-fixed by immersion in 4% paraformaldehyde/PBS overnight and then transferred to 30% (w/v) sucrose. Brains were coronally sectioned at 40 µm by slicing microtome and stored in antifreeze solution at +4° C. for posterior histological analysis.

### Results

### Increases in the Levels of CB2 mRNA in the Liver of MPSIIIA Mice

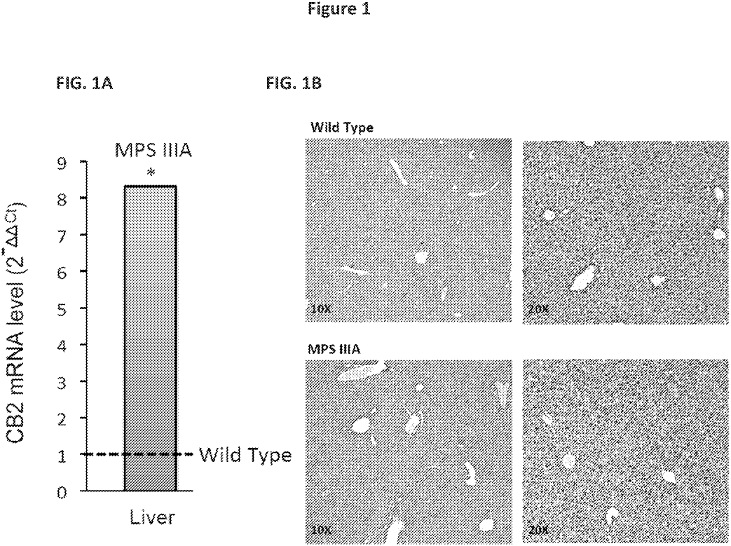

We measured the mRNA levels of CB2 by qPCR in extracts of liver tissue from wild type and MPSIIIA mice at 10 months of age, and found significant increases in CB2 mRNA expression (8 fold increase) (FIG. 1A).

### Increases in the Levels of CB2 Protein in the Liver and Brains of MPSIIIA Mice

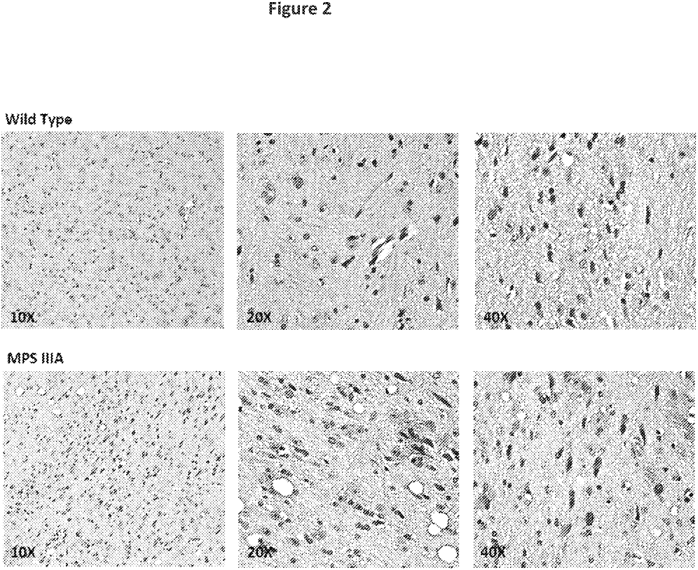

We measured the protein levels of CB2 by immunofluorescence in the liver (FIG. 1B) and cortex (FIG. 2A) of wt and MPSIIIA mice at 10 months of age and observed marked increases in CB2 protein expression in the tissues of these organs from MPSIIIA mice as compared to the amounts in wild type mice.

### Conclusions

Expression of the endocannabinoid receptor CB2 is markedly increased in MPSIIIA mice in both the brain and liver.

### Example 2

### Alterations of the Endocannabinoid Receptor Cb2 in the Liver, Spleen, And Brain of Farber Mice, a Model for Human Farber Disease

### Background

Farber disease (acid ceramidase deficiency, lipogranulomatosis) is a rare lysosomal storage disorder caused by mutations in the lysosomal acid ceramidase (ASAH1) gene. Acid ceramidase is responsible for the degradation of ceramide to sphingosine and fatty acid, and a deficiency of acid ceramidase activity leads to the accumulation of ceramide and resultant abnormalities in the joints, liver, throat, tissues and central nervous system. Individuals with Farber disease often exhibit a characteristic “hoarse” cry or voice due to the presence of lesions on the larynx. The involvement of other organ systems in some of these patients, including the lung, liver, spleen and central nervous system (CNS), also may be present. For most children, the disease is fatal by the age of two. And, in some severe forms of the disease that include enlargement of the liver and spleen, death typically occurs within 6 months of age. A variant form of Farber disease, also known as Spinal Muscular Atrophy with Progressive Myoclonic Epilepsy (SMA-PME), presents with primarily neurologic symptoms.

The Farber mouse is a well-established model for human Farber disease. These mice were generated to carry a single-nucleotide mutation in the ASAH1 gene that was identified in human Farber Disease patients (P361R), and these mice exhibit hallmark features of the human disease including deficient acid ceramidase activity, ceramide accumulation, demonstrated Farber Disease manifestations, and death within 7-13 weeks. (Alayoubi AM, et al., EMBO Mol Med. 2013 Jun;5(6):827-42; incorporated herein by reference in its entirety).

The present study analyzed CB2 cannabinoid receptor expression in the liver, spleen, cerebellum, and cortex of 8.5 week old Farber mice. The Farber Mice we utilized were in the C57BL/6 strain, and in all experiments, mice were fed regular diet and provided water ad libitum from lixits. Environmental controls for the animal room were performed/maintained as described in Example 1.

### Results

### Increases in the Levels of CB2 Protein Levels

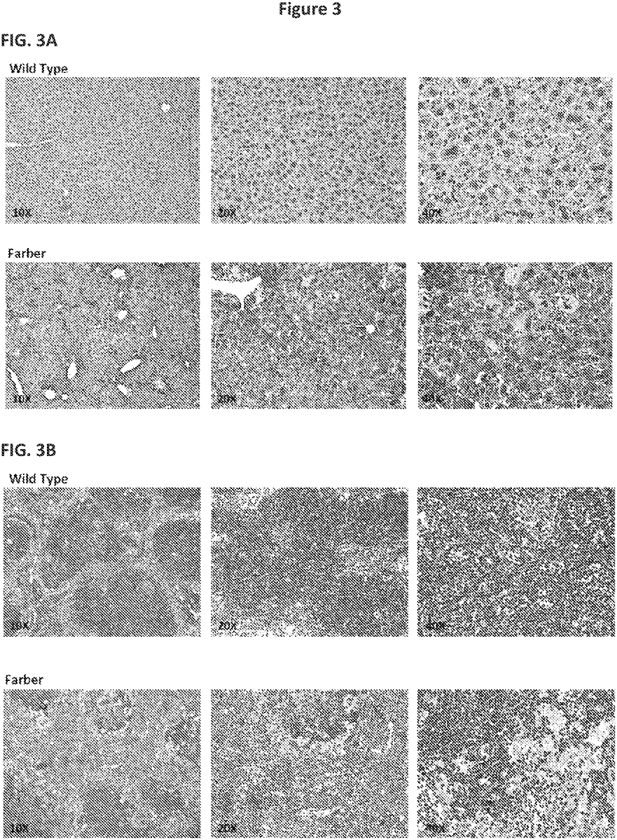

We analyzed protein expression levels of the CB2 cannabinoid receptor in the liver (FIG. 3A), spleen (FIG. 3B), cerebellum (FIG. 3C), and cortex (FIG. 3C) of 8.5 week old Farber mice by immunofluorescence using the methods described in Example 1. In all tissues, marked increases in CB2 expression was observed in Farber mice as compared to wild type controls.

### Conclusions

As was the case for the MPSIIIA mice studied in Example 1, expression of the endocannabinoid receptor CB2 is markedly increased in the brain and periphery of Farber mice.

### Example 3

### Activation of CB2 Prolongs the Life of Farber Mice and Ameliorates Disease Associated Weight Loss

The high expression of CB2 in the diverse lysosomal diseases MPSIIIA and Farber Disease suggests that activation of this receptor might be an optimal therapeutic target, as agonists of CB2 have been shown to inhibit inflammation, which is one of the hallmark symptoms common to most if not all lysosomal storage disease and disorder. In addition, agonists of CB2 also have been shown to prevent or slow neurodegeneration, a neurologic hallmark of all or most lysosomal storage diseases with neurologic symptoms. Accordingly, in this Example we treated Farber mice with the CB2 selective agonist JWH133.

Two Farber mouse groups were studied (n=12 mice per group). In the treatment group mice were injected (i.p.) three times a week with 5 mg/kg of JWH133 dissolved in DMSO, starting at 2 weeks of age. In the control group they were injected with DMSO alone. Weight was monitored daily. All mice in the control group were allowed to die naturally or were euthanized if they lost >10% body weight within a week (as per IACUC protocols).

### Results

Farber Mice treated with the CB2 agonist JWH133 survived longer than vehicle control treated mice. All 12 mice in the vehicle control group were dead by day 44. In contrast, in the treatment group only 1 of the mice died naturally (on day 47) - the 11 remaining mice were euthanized at day 58, and organs and blood were collected and stored for future analysis. In addition, throughout the study the weight of the mice treated with JWH133 was significantly greater than Farber Mice treated with vehicle alone. Thus, these data confirm that Farber disease, which displays elevated levels of CB2 expression may be effectively treated with a CB2 agonist. Moreover, our data also confirmed that CB2 expression is upregulated in another lysosomal storage disease, MPSIIIA, which is caused by an entirely different mechanism of action than in Farber disease. Without being limited by conjecture, we believe that this is due to the underlying inflammation and microglia activation that occurs in each of these diseases and in most of the lysosomal storage diseases and disorders. Thus, these data strongly support that diseases that are caused by or that are accompanied with increases in CB2 expression share a common mechanism and will be commonly treatable with agonists of the CB2 receptor.

### Example 4

### Using CB2 as a Biomarker for Monitoring the Efficacy of Enzyme Replacement Therapy

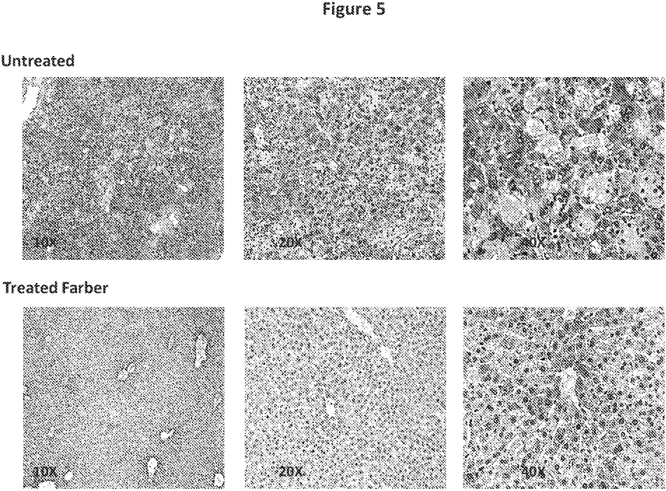

Ten 2.5-week-old Farber disease mice were treated once weekly with recombinant acid ceramidase by intraperitoneal injection at a dose of 10 mg/kg. After 6 injections the 8.5-week-old mice were euthanized and their organs collected and processed for immunohistochemical analysis of CB2 expression. As evident from the representative images in FIG. 5, CB2 expression was significantly reduced in mice treated by enzyme replacement therapy compared to untreated Farber mice, consistent with the reduced inflammation. Thus, CB2 can be used as a biomarker to monitor the efficacy of treatment for Farber disease and other lysosomal storage disorders.

### Example 5

### Reducing Inflammation Using CB2 Agonist

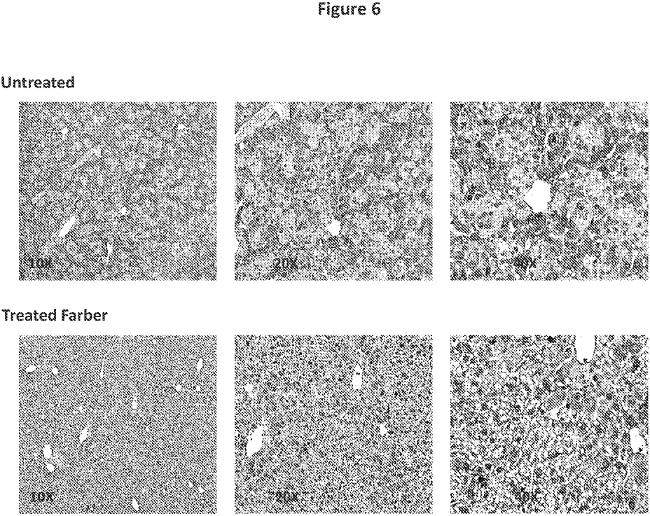

Ten 2.5-week-old Farber disease mice were treated 3x a week by intraperitoneal injection of JWH133 at a dose of 5 mg/kg. After 6 weeks the 8.5-week-old mice were euthanized and their organs collected and processed for immunohistochemical analysis of CB2 expression. As evident from the representative image in FIG. 6, CB2 expression was significantly reduced in mice treated by the CB2 agonist JWH133 compared to untreated Farber mice, consistent with the reduced inflammation. Thus, CB2 agonists, such as JWH133, can be used to treat Farber disease and other lysosomal storage disorders.

### Example 6

### Reducing MCP-1 Expression in Liver and Plasma of Mouse Model Using CB2 Agonist

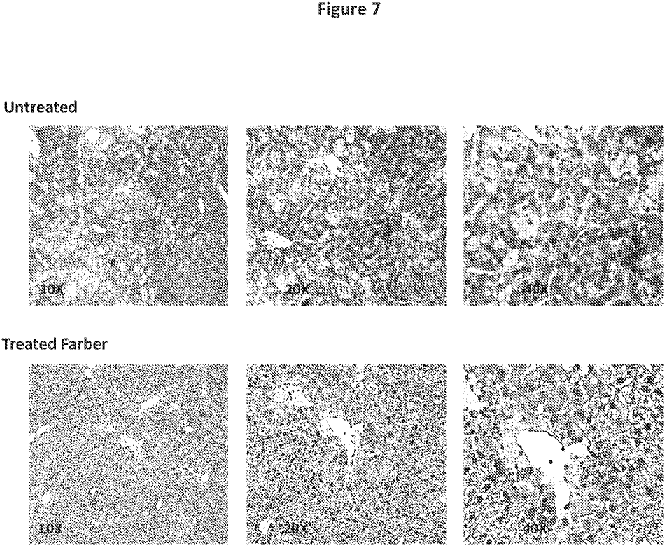

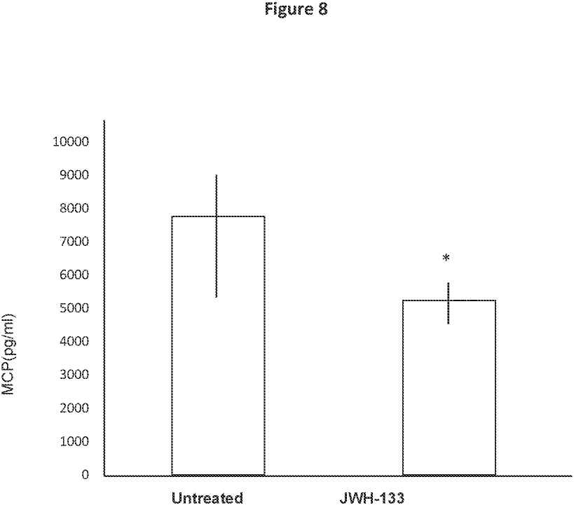

Ten 2.5-week-old Farber disease mice were treated 3x a week by intraperitoneal injection of JWH133 at a dose of 5 mg/kg. After 6 weeks the 8.5-week-old mice were euthanized and their organs and plasma collected and processed for immunohistochemical analysis of MCP-1 expression. MCP-1 expression was significantly reduced in the liver (FIG. 7) and plasma (FIG. 8) of mice treated by the CB2 agonist JWH133 compared to untreated Farber mice, consistent with the reduced inflammation. Thus, CB2 agonists, such as JWH133, can be used to treat Farber disease and other lysosomal storage disorders (* = p<0.01).

### Example 7

### reducing MCP-1 Expression in MPSIIIA Patient Sample Using CB2 Agonist

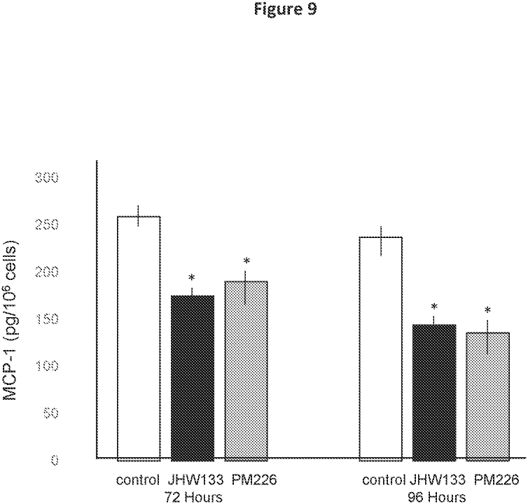

Skin fibroblasts from a patient with MPS IIIA were grown to ~70% confluency and the CB2 agonists JHW133 and PM226 were added to the culture media for 72 or 96 hr at a dose of 3.2 micromoles/ml (black and gray bars, respectively). The levels of MCP-1 in the media were then measured by ELISA ans shown in FIG. 9. Control: cultures without drug added (white bars). Both CB2 agonists were able to reduce the levels of MCP-1 released into the culture media. Bar heights indicate the mean values from three independent experiments. Vertical lines indicate the standard error of the mean. * = p < 0.01 comparing with and without drug.

### NUMBERED EMBODIMENTS OF THE DISCLOSURE

Other subject matter contemplated by the present disclosure is set out in the following numbered embodiments:

1. A method for reducing inflammation in a subject that has a lysosomal storage disease or disorder comprising, selectively or specifically activating the CB2 cannabinoid receptor in the subject.

2. The method of embodiment 1, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

3. The method of embodiment 2, wherein the lipidoses is selected Gaucher disease, Fabry disease, Farber Disease, Krabbe Disease, Metachromatic Leukodystrophy, Acid Spingomyelinase Deficiency (ASMD), Niemann-Pick Disease Type C, Gangliosidoses 1, and Gangliosidoses 2.

4. The method of embodiment 2, wherein the mucopolysaccharidoses is selected from MPS types I, II, III, IV, VI, VII and IX.

5. The method of embodiment 2, wherein the Mucolipidoses is selected from Mucolipidoses I, Mucolipidoses 2, Mucolipidoses 3 and Mucolipidoses 4.

6. The method of embodiment 2, wherein the glycogen storage disease is Pompe Disease.

7. The method of embodiment 2, wherein the carbohydrate storage diseases is selected from Mannosidosis and Sialidosis.

8. The method of embodiment 2, wherein the transport diseases is selected from Cystinosis and Salla Disease.

9. The method of embodiment 2, wherein the neuronal ceroid lipofuscinosis is Batten’s disease.

10. The method of embodiment 1, wherein the lysosomal storage disease or disorder is Farber Disease, or mucopolysaccharidosis type IIIA (MPS IIIA).

11. The method of any one of the preceding embodiments, wherein the inflammation is modulated in the subject’s periphery.

12. The method of any one of the preceding embodiments, wherein the inflammation is modulated by activating the CB2 receptor on peripheral macrophages in the subject.

13. The method of any one of the preceding embodiments, wherein the inflammation is modulated in the subject’s CNS.

14. The method of embodiment 13, wherein the inflammation is modulated in the subject’s CNS by activating the CB2 receptor on microglia in the subject.

15. The method of embodiment 13 or 14, wherein the inflammation is modulated in the subject’s CNS by inhibiting microglia activation.

16. The method of any one of the preceding embodiments, wherein the CB2 cannabinoid receptor is activated by a selective or specific agonist of the CB2 cannabinoid receptor.

17. The method of embodiment 16, wherein the selective or specific CB2 agonist is administered to the subject as a pharmaceutical composition.

18. The method of embodiment 17, wherein the pharmaceutical composition comprises the selective or specific agonist of the CB2 cannabinoid receptor and one or more pharmaceutically acceptable salts, excipients or vehicles.

19. The method of embodiment 17 or 18, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

20. The method of any one of embodiments 16-19, wherein the selective or specific agonist of the CB2 cannabinoid receptor selected from HU-308, JWH-015, GW-405833 (L-768242), AM-1241, JWH-133, GW-842166X, and the following compounds:

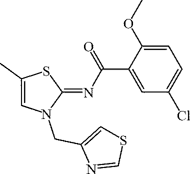 [SMILES: COc1ccc(Cl)cc1C(=O)/N=c1\sc(C)cn1Cc1cscn1]

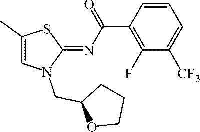 [SMILES: Cc1cn(C[C@H]2CCCO2)/c(=N/C(=O)c2cccc(C(F)(F)F)c2F)s1]

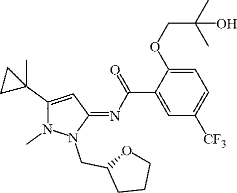

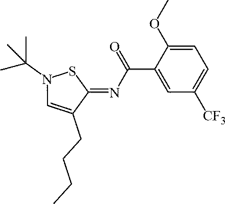 [SMILES: CCCCc1cn(C(C)(C)C)s/c1=N\C(=O)c1cc(C(F)(F)F)ccc1OC]

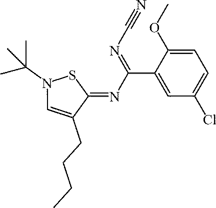 [SMILES: CCCCc1cn(C(C)(C)C)s/c1=N\C(=N\C#N)c1cc(Cl)ccc1OC]

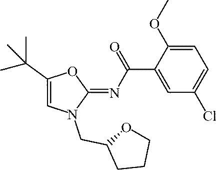

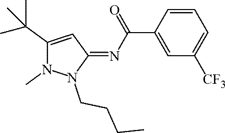 [SMILES: CCCCn1/c(=N/C(=O)c2cccc(C(F)(F)F)c2)cc(C(C)(C)C)n1C]

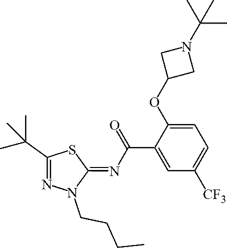 [SMILES: CCCCn1nc(C(C)(C)C)s/c1=N\C(=O)c1cc(C(F)(F)F)ccc1OC1CN(C(C)(C)C)C1]

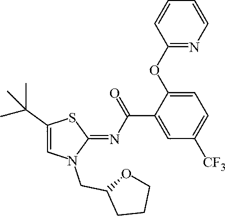

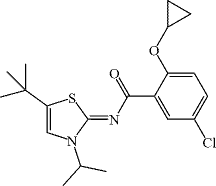 [SMILES: CC(C)n1cc(C(C)(C)C)s/c1=N\C(=O)c1cc(Cl)ccc1OC1CC1]

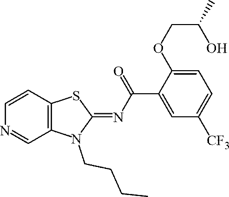 [SMILES: CCCCn1/c(=N/C(=O)c2cc(C(F)(F)F)ccc2OC[C@H](C)O)sc2ccncc21]

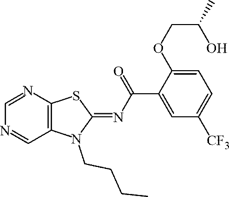 [SMILES: CCCCn1/c(=N/C(=O)c2cc(C(F)(F)F)ccc2OC[C@H](C)O)sc2ncncc21]

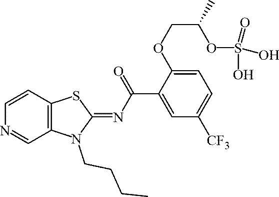

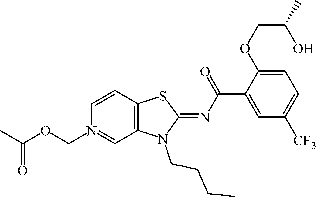

21. The method of any one of the preceding embodiments, wherein the CB2 activation treats the lysosomal storage disease or disorder or alleviates one or more symptom of the lysosomal storage disease or disorder.

22. The method of any one of the preceding embodiments, wherein the method prevents neuronal death associated with the lysosomal storage disease or disorder.

23. The method of any one of the preceding embodiments, wherein the method improves the subject’s cognition, learning and/or memory.

24. A method of treating a subject that has a lysosomal storage disease or disorder comprising, administering to the subject a selective or specific agonist of the CB2 cannabinoid receptor.

25. A method of inhibiting a loss of motor skills, cognitive decline, and/or systemic organ pathology associated with a lysosomal storage disease or disorder comprising, administering to the subject a selective or specific agonist of the CB2 cannabinoid receptor.

26. The method of embodiment 24 or 25, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

27. The method of embodiment 26, wherein the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2.

28. The method of embodiment 26, wherein the mucopolysaccharidoses is selected from MPS types I, II, III, IV, VI, VII and IX.

29. The method of embodiment 26, wherein the Mucolipidoses is selected from Mucolipidoses I, Mucolipidoses 2, Mucolipidoses 3 and Mucolipidoses 4.

30. The method of embodiment 26, wherein the glycogen storage disease is Pompe Disease.

31. The method of embodiment 26, wherein the carbohydrate storage diseases is selected from Mannosidosis and Sialidosis.

32. The method of embodiment 26, wherein the transport diseases is selected from Cystinosis and Salla Disease.

33. The method of embodiment 26, wherein the neuronal ceroid lipofuscinosis is Batten’s disease.

34. The method of embodiment 24 or 25, wherein the lysosomal storage disease or disorder is Farber Disease or mucopolysaccharidosis type IIIA (MPS IIIA).

35. The method of any one of embodiments 24-34, wherein the selective or specific agonist of the CB2 cannabinoid receptor is administered to the subject as a pharmaceutical composition.

36. The method of embodiment 35, wherein the pharmaceutical composition comprises the selective or specific agonist of the CB2 cannabinoid receptor and one or more pharmaceutically acceptable salts, excipients or vehicles.

37. The method of embodiment 35 or 36, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

38. The method of any one of embodiments 24-37, wherein the selective or specific agonist of the CB2 cannabinoid receptor selected from HU-308, JWH-015, GW-405833 (L-768242), AM-1241, JWH-133, GW-842166X, and the following compounds:

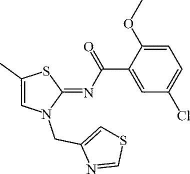 [SMILES: COc1ccc(Cl)cc1C(=O)/N=c1\sc(C)cn1Cc1cscn1]

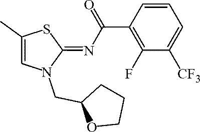 [SMILES: Cc1cn(C[C@H]2CCCO2)/c(=N/C(=O)c2cccc(C(F)(F)F)c2F)s1]

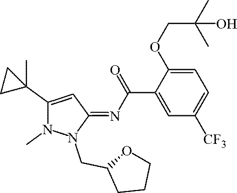

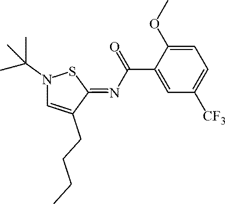 [SMILES: CCCCc1cn(C(C)(C)C)s/c1=N\C(=O)c1cc(C(F)(F)F)ccc1OC]

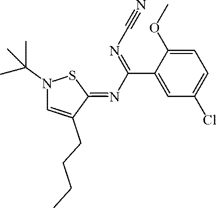 [SMILES: CCCCc1cn(C(C)(C)C)s/c1=N\C(=N\C#N)c1cc(Cl)ccc1OC]

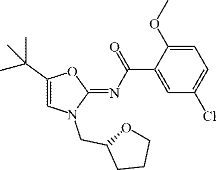

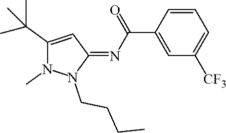 [SMILES: CCCCn1/c(=N/C(=O)c2cccc(C(F)(F)F)c2)cc(C(C)(C)C)n1C]

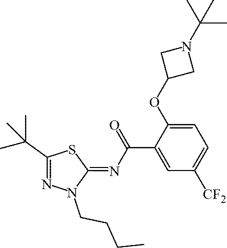

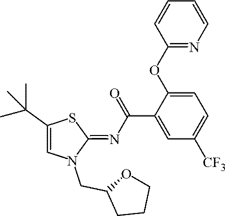

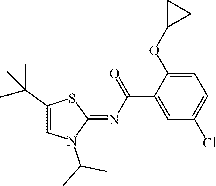 [SMILES: CC(C)n1cc(C(C)(C)C)s/c1=N\C(=O)c1cc(Cl)ccc1OC1CC1]

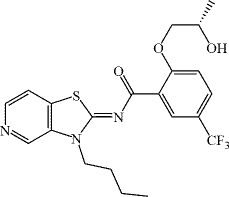 [SMILES: CCCCn1/c(=N/C(=O)c2cc(C(F)(F)F)ccc2OC[C@H](C)O)sc2ccncc21]

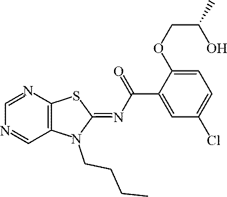 [SMILES: CCCCn1/c(=N/C(=O)c2cc(Cl)ccc2OC[C@H](C)O)sc2ncncc21]

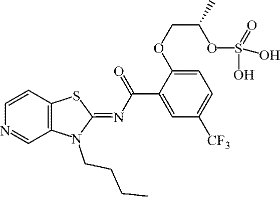

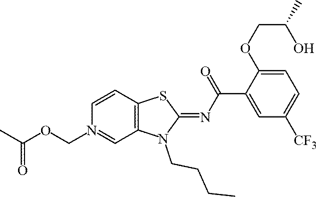

39. The method of any one of embodiments 24-38, wherein the method treats the lysosomal storage disease or disorder or results in the improvement of one or more symptoms associated with the lysosomal storage disease or disorder.

40. The method of any one of embodiments 24-39, wherein the method prevents neuronal death associated with the lysosomal storage disease or disorder.

41. The method of any one of embodiments 24-40, wherein the method improves the subject’s cognition.

42. The method of any one of embodiments 24-41 wherein the method improves the subject’s learning and/or memory.

43. The method of any one of embodiments 24-42, wherein the method reduces inflammation levels in the subject.

44. The method of any one of embodiments 24-43, wherein the method treats the brain or CNS of the subject.

45. The method of any one of embodiments 24-44, wherein the method activates CB2 on microglia in the brain of the subject.

46. The method of any one of embodiments 24-45, wherein the method reduces activation of microglia in the brain of the subject.

47. The method of any one of embodiments 24-46, wherein the method treats the reticuloendothelial system.

48. The method of any one of embodiments 24-47, wherein the method treats the skeletal system.

49. The method of any one of embodiments 24-48, wherein the method results in reduced inflammation levels in an organ other than the brain.

50. The method of embodiment 49, wherein the organ other than the brain is selected from the liver, spleen, lungs, adrenal gland, kidney, heart, articular cartilage, articular joint space, and bone marrow.

51. The method of any one of embodiments 24-50, wherein the lifespan of the subject is increased as compared to the expected lifespan of an individual with the same lysosomal storage disease or disorder that has not been administered the selective agonist of the CB2 cannabinoid receptor.

52. The method of any one of embodiments 24-51 wherein the method further comprises administering one or more additional therapeutic agents.

53. The method of embodiment 52, wherein the additional therapeutic agent comprises an enzyme replacement therapy, gene therapy, substrate reduction therapy, chaperone therapy, or a small molecule therapy.

54. The method of any one of embodiments 1-23, further comprising activating the CB1 cannabinoid receptor.

55. The method of any one of embodiments 34-53, further comprising administering a direct or indirect agonist of the CB1 cannabinoid receptor, wherein the indirect agonist is optionally a FAAHi.

56. A method of inhibiting macrophage activation in a subject with a lysosomal storage disease or disorder comprising administering to the subject a selective or specific agonist of the CB2 cannabinoid receptor.

57. The method of embodiment 56, wherein the macrophage is a peripheral macrophage in the subject.

58. The method of 56, wherein the macrophage is a microglia in the central nervous system (CNS) of the subject.

59. The method of any one of embodiments 56-58, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

60. The method of embodiment 59, wherein the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2.

61. The method of embodiment 59, wherein the mucopolysaccharidoses is selected from MPS types I, II, III, IV, VI, VII and IX.

62. The method of embodiment 59, wherein the Mucolipidoses is selected from Mucolipidoses I, Mucolipidoses 2, Mucolipidoses 3 and Mucolipidoses 4.

63. The method of embodiment 59, wherein the glycogen storage disease is Pompe Disease.

64. The method of embodiment 59, wherein the carbohydrate storage diseases is selected from Mannosidosis and Sialidosis.

65. The method of embodiment 59, wherein the transport diseases is selected from Cystinosis and Salla Disease.

66. The method of embodiment 59, wherein the neuronal ceroid lipofuscinosis is Batten’s disease.

67. The method of embodiment 56, wherein the lysosomal storage disease or disorder is Farber Disease or mucopolysaccharidosis type IIIA (MPS IIIA).

68. The method of any one of embodiments 56-67, wherein the selective or specific agonist of the CB2 cannabinoid receptor is administered to the subject as a pharmaceutical composition.

69. The method of embodiment 68, wherein the pharmaceutical composition comprises the selective or specific agonist of the CB2 cannabinoid receptor and one or more pharmaceutically acceptable salts, excipients or vehicles.

70. The method of embodiment 68 or 69, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

71. The method of any one of embodiments 56-70, wherein the selective or specific agonist of the CB2 cannabinoid receptor selected from HU-308, JWH-015, GW-405833 (L-768562), AM-1561, JWH-133, GW-842166X, and the following compounds:

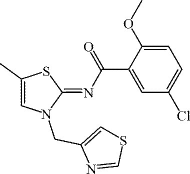 [SMILES: COc1ccc(Cl)cc1C(=O)/N=c1\sc(C)cn1Cc1cscn1]

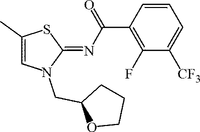 [SMILES: Cc1cn(C[C@H]2CCCO2)/c(=N/C(=O)c2cccc(C(F)(F)F)c2F)s1]

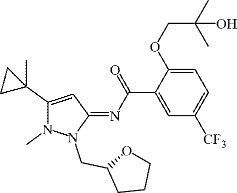

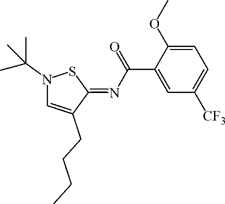 [SMILES: CCCCc1cn(C(C)(C)C)s/c1=N\C(=O)c1cc(C(F)(F)F)ccc1OC]

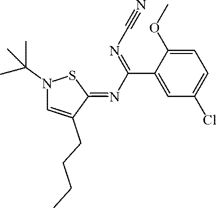 [SMILES: CCCCc1cn(C(C)(C)C)s/c1=N\C(=N\C#N)c1cc(Cl)ccc1OC]

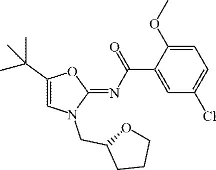

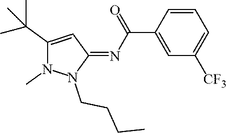 [SMILES: CCCCn1/c(=N/C(=O)c2cccc(C(F)(F)F)c2)cc(C(C)(C)C)n1C]

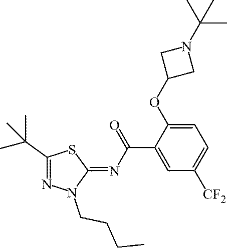

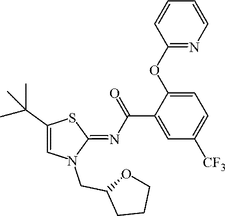

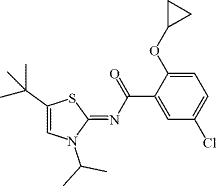 [SMILES: CC(C)n1cc(C(C)(C)C)s/c1=N\C(=O)c1cc(Cl)ccc1OC1CC1]

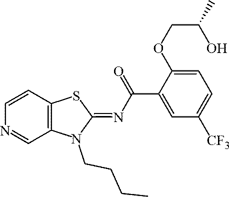 [SMILES: CCCCn1/c(=N/C(=O)c2cc(C(F)(F)F)ccc2OC[C@H](C)O)sc2ccncc21]

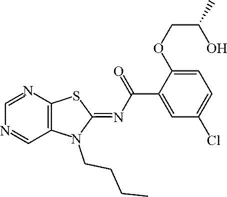 [SMILES: CCCCn1/c(=N/C(=O)c2cc(Cl)ccc2OC[C@H](C)O)sc2ncncc21]

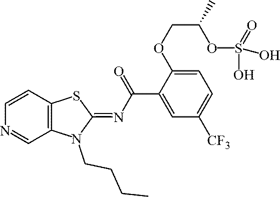

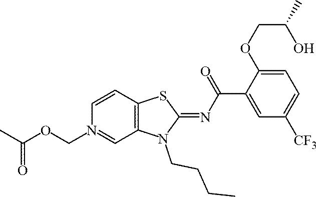

72. The method of any one of embodiments 56-71, wherein the method treats the lysosomal storage disease or disorder or results in the improvement of one or more symptoms associated with the lysosomal storage disease or disorder.

73. The method of any one of embodiments 56-72, wherein the method prevents neuronal death associated with the lysosomal storage disease or disorder.

74. The method of any one of embodiments 56-73, wherein the method improves the subject’s cognition.

75. The method of any one of embodiments 56-74 wherein the method improves the subject’s learning and/or memory.

76. The method of any one of embodiments 56-75, wherein the method reduces inflammation levels in the subject.

77. The method of any one of embodiments 56-76, wherein the method activates CB2 in the brain or CNS of the subject.

78. The method of any one of embodiments 56-77, wherein the method activates CB2 on microglia in the brain of the subject.

79. The method of any one of embodiments 56-78, wherein the method treats the reticuloendothelial system.

80. The method of any one of embodiments 56-79, wherein the method treats the skeletal system.

81. The method of any one of embodiments 56-80, wherein the method results in reduced inflammation levels in an organ other than the brain.

82. The method of embodiment 81, wherein the organ other than the brain is selected from the liver, spleen, lungs, adrenal gland, kidney, heart, articular cartilage, articular joint space, and bone marrow.

83. The method of any one of embodiments 56-82, wherein the lifespan of the subject is increased as compared to the expected lifespan of an individual with the same lysosomal storage disease or disorder that has not been administered the selective agonist of the CB2 cannabinoid receptor.

84. The method of any one of embodiments 56-83 wherein the method further comprises administering one or more additional therapeutic agents.

85. The method of embodiment 84, wherein the additional therapeutic agent comprises an enzyme replacement therapy, gene therapy, substrate reduction therapy, chaperone therapy, or a small molecule therapy.

86. The method of any one of embodiments 56-85, further comprising administering a direct or indirect agonist of the CB1 cannabinoid receptor, wherein the indirect agonist is optionally a FAAHi.

87. A method of reducing at least one inflammatory cytokine or chemokine in a subject with a lysosomal storage disease or disorder comprising selectively or specifically activating the CB2 cannabinoid receptor.

88. The method of embodiment 87, wherein the inflammatory cytokine or chemokine is selected from MCP-1, MIP1 alpha, TNF alpha, IL-1beta, IL-8, GM-CSF.

89. The method of embodiment 87 or 88, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

90. The method of embodiment 89, wherein the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2.

91. The method of embodiment 89, wherein the mucopolysaccharidoses is selected from MPS types I, II, III, IV, VI, VII and IX.

92. The method of embodiment 89, wherein the Mucolipidoses is selected from Mucolipidoses I, Mucolipidoses 2, Mucolipidoses 3 and Mucolipidoses 4.

93. The method of embodiment 89, wherein the glycogen storage disease is Pompe Disease.

94. The method of embodiment 89, wherein the carbohydrate storage diseases is selected from Mannosidosis and Sialidosis.

95. The method of embodiment 89, wherein the transport diseases is selected from Cystinosis and Salla Disease.

96. The method of embodiment 89, wherein the neuronal ceroid lipofuscinosis is Batten’s disease.

97. The method of embodiment 87, wherein the lysosomal storage disease or disorder isFarber Diseaseor mucopolysaccharidosis type IIIA (MPS IIIA).

98. The method of any one of embodiments 87-97, wherein the selective or specific agonist of the CB2 cannabinoid receptor is administered to the subject as a pharmaceutical composition.

99. The method of embodiment 98, wherein the pharmaceutical composition comprises the selective or specific agonist of the CB2 cannabinoid receptor and one or more pharmaceutically acceptable salts, excipients or vehicles.

100. The method of embodiment 98 or 99, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

101. The method of any one of embodiments 87-100, wherein the selective or specific agonist of the CB2 cannabinoid receptor selected from HU-308, JWH-015, GW-405833 (L-798872), AM-1871, JWH-133, GW-842166X, and the following compounds:

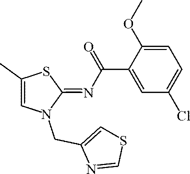 [SMILES: COc1ccc(Cl)cc1C(=O)/N=c1\sc(C)cn1Cc1cscn1]

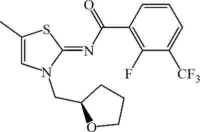 [SMILES: Cc1cn(C[C@H]2CCCO2)/c(=N/C(=O)c2cccc(C(F)(F)F)c2F)s1]

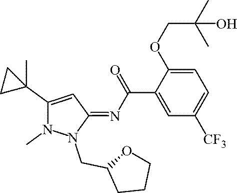

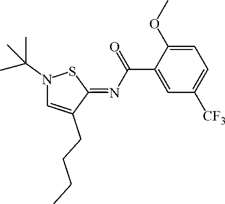 [SMILES: CCCCc1cn(C(C)(C)C)s/c1=N\C(=O)c1cc(C(F)(F)F)ccc1OC]

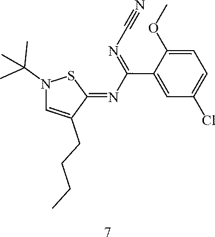

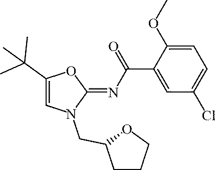

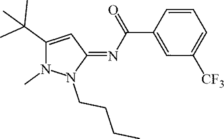 [SMILES: CCCCn1/c(=N/C(=O)c2cccc(C(F)(F)F)c2)cc(C(C)(C)C)n1C]

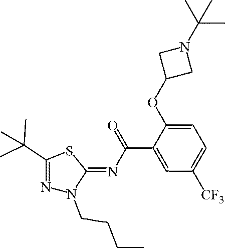 [SMILES: CCCCn1nc(C(C)(C)C)s/c1=N\C(=O)c1cc(C(F)(F)F)ccc1OC1CN(C(C)(C)C)C1]

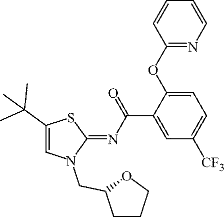

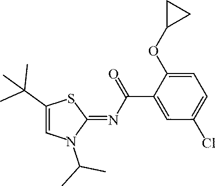 [SMILES: CC(C)n1cc(C(C)(C)C)s/c1=N\C(=O)c1cc(Cl)ccc1OC1CC1]

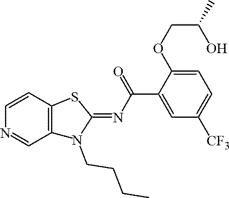 [SMILES: CCCCn1/c(=N/C(=O)c2cc(C(F)(F)F)ccc2OC[C@H](C)O)sc2ccncc21]

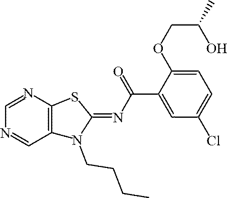 [SMILES: CCCCn1/c(=N/C(=O)c2cc(Cl)ccc2OC[C@H](C)O)sc2ncncc21]

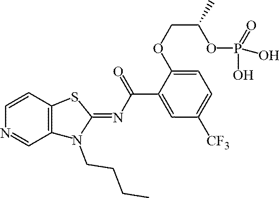 [SMILES: CCCCn1/c(=N/C(=O)c2cc(C(F)(F)F)ccc2OC[C@H](C)OP(=O)(O)O)sc2ccncc21]

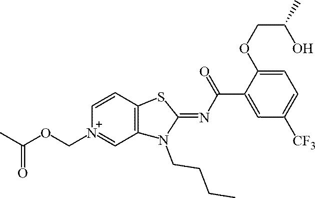 [SMILES: CCCCn1/c(=N/C(=O)c2cc(C(F)(F)F)ccc2OC[C@H](C)O)sc2cc[n+](COC(C)=O)cc21]

102. The method of any one of embodiments 87-101, wherein the method treats the lysosomal storage disease or disorder or results in the improvement of one or more symptoms associated with the lysosomal storage disease or disorder.

103. The method of any one of embodiments 87-102, wherein the method prevents neuronal death associated with the lysosomal storage disease or disorder.

104. The method of any one of embodiments 87-103, wherein the method improves the subject’s cognition.

105. The method of any one of embodiments 87-104 wherein the method improves the subject’s learning and/or memory.

106. The method of any one of embodiments 87-105, wherein the method reduces inflammation levels in the subject.

107. The method of any one of embodiments 87-106, wherein the method reduces inflammation levels in the brain or CNS of the subject.

108. The method of any one of embodiments 87-106, wherein the method activates CB2 on microglia in the brain of the subject.

109. The method of any one of embodiments 87-108, wherein the method treats the reticuloendothelial system.

110. The method of any one of embodiments 87-109, wherein the method treats the skeletal system.

111. The method of any one of embodiments 87-110, wherein the method results in reduced inflammation levels in an organ other than the brain.

112. The method of embodiment 111, wherein the organ other than the brain is selected from the liver, spleen, lungs, adrenal gland, kidney, heart, articular cartilage, articular joint space, and bone marrow.

113. The method of any one of embodiments 87-112, wherein the lifespan of the subject is increased as compared to the expected lifespan of an individual with the same lysosomal storage disease or disorder that has not been administered the selective agonist of the CB2 cannabinoid receptor.

114. The method of any one of embodiments 87-113 wherein the method further comprises administering one or more additional therapeutic agents.

115. The method of embodiment 114, wherein the additional therapeutic agent comprises an enzyme replacement therapy, gene therapy, substrate reduction therapy, chaperone therapy, or a small molecule therapy.

116. The method of any one of embodiments 87-115, further comprising administering a direct or indirect agonist of the CB1 cannabinoid receptor.

117. A composition comprising an agent capable of selectively activating the CB2 cannabinoid receptor, for use in a method of treating a lysosomal disease or disorder.

118. The composition of embodiment 117, further comprising an agent capable of directly or indirectly activating the CB1 cannabinoid receptor.

119. A pharmaceutical composition comprising the composition of embodiment 117 or 118 and and one or more pharmaceutically acceptable salts, excipients or vehicles.

120. The pharmaceutical composition of embodiment 119, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

121. A method of reducing beta amyloid and or tau protein levels in the brain of subject with a lysosomal storage disease or disorder comprising administering to the subject a selective or specific agonist of the CB2 cannabinoid receptor.

122. The method of embodiment 121, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

123. The method of embodiment 122, wherein the lipidoses is selected Gaucher disease, Fabry disease, Farber Disease, Krabbe Disease, Metachromatic Leukodystrophy, Acid Sphingomyelinase Deficiency (ASMD), Niemann-Pick Disease Type C, Gangliosidoses 1, and Gangliosidoses 2.

124. The method of embodiment 122, wherein the mucopolysaccharidoses is selected from MPS types I, II, III, IV, VI, VII and IX.

125. The method of embodiment 122, wherein the Mucolipidoses is selected from Mucolipidoses I, Mucolipidoses 2, Mucolipidoses 3 and Mucolipidoses 4.

126. The method of embodiment 122, wherein the glycogen storage disease is Pompe Disease.

127. The method of embodiment 122, wherein the carbohydrate storage diseases is selected from Mannosidosis and Sialidosis.

128. The method of embodiment 122, wherein the ttransport diseases is selected from Cystinosis and Salla Disease.

129. The method of embodiment 122, wherein the neuronal ceroid lipofuscinosis is Batten’s disease.

130. The method of embodiment 121, wherein the lysosomal storage disease or disorder is Farber Diseaseor mucopolysaccharidosis type IIIA (MPS IIIA).

131. The method of any one of embodiments 121-130, wherein the selective or specific agonist of the CB2 cannabinoid receptor is administered to the subject as a pharmaceutical composition.

132. The method of embodiment 131, wherein the pharmaceutical composition comprises the selective or specific agonist of the CB2 cannabinoid receptor and one or more pharmaceutically acceptable salts, excipients or vehicles.

133. The method of embodiment 131 or 132, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

134. The method of any one of embodiments 121-133, wherein the selective or specific agonist of the CB2 cannabinoid receptor selected from HU-308, JWH-015, GW-405833 (L-7681212), AM-11211, JWH-133, GW-842166X, and the following compounds:

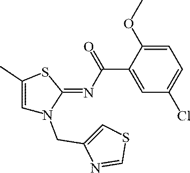 [SMILES: COc1ccc(Cl)cc1C(=O)/N=c1\sc(C)cn1Cc1cscn1]

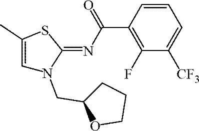 [SMILES: Cc1cn(C[C@H]2CCCO2)/c(=N/C(=O)c2cccc(C(F)(F)F)c2F)s1]

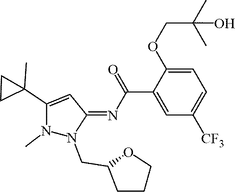

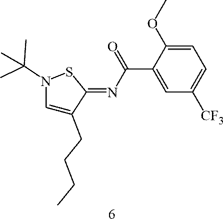

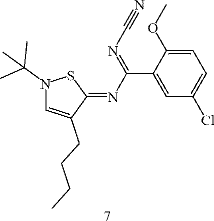

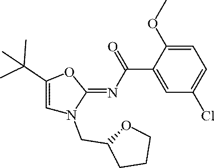

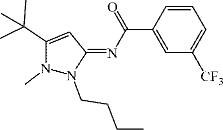 [SMILES: CCCCn1/c(=N/C(=O)c2cccc(C(F)(F)F)c2)cc(C(C)(C)C)n1C]

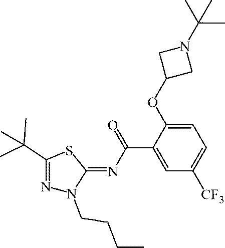 [SMILES: CCCCn1nc(C(C)(C)C)s/c1=N\C(=O)c1cc(C(F)(F)F)ccc1OC1CN(C(C)(C)C)C1]

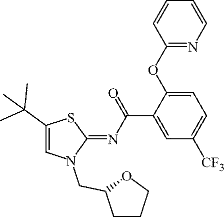

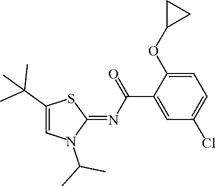 [SMILES: CC(C)n1cc(C(C)(C)C)s/c1=N\C(=O)c1cc(Cl)ccc1OC1CC1]

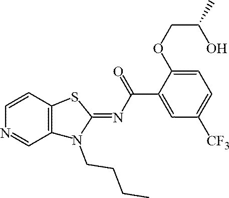 [SMILES: CCCCn1/c(=N/C(=O)c2cc(C(F)(F)F)ccc2OC[C@H](C)O)sc2ccncc21]

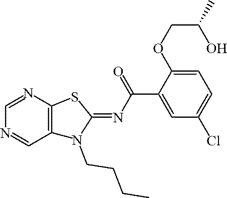 [SMILES: CCCCn1/c(=N/C(=O)c2cc(Cl)ccc2OC[C@H](C)O)sc2ncncc21]

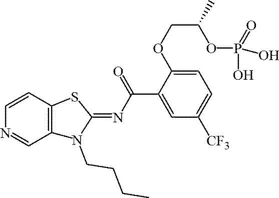 [SMILES: CCCCn1/c(=N/C(=O)c2cc(C(F)(F)F)ccc2OC[C@H](C)OP(=O)(O)O)sc2ccncc21]

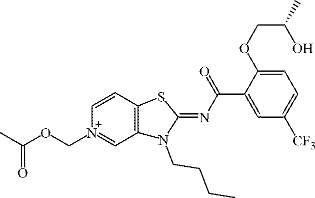 [SMILES: CCCCn1/c(=N/C(=O)c2cc(C(F)(F)F)ccc2OC[C@H](C)O)sc2cc[n+](COC(C)=O)cc21]

135. The method of any one of embodiments 121-134, wherein the method treats the lysosomal storage disease or disorder or results in the improvement of one or more symptoms associated with the lysosomal storage disease or disorder.

136. The method of any one of embodiments 121-135, wherein the method prevents neuronal death associated with the lysosomal storage disease or disorder.

137. The method of any one of embodiments 121-136, wherein the method improves the subject’s cognition.

138. The method of any one of embodiments 121-137 wherein the method improves the subject’s learning and/or memory.

139. The method of any one of embodiments 121-138, wherein the method reduces inflammation levels in the subject.

140. The method of any one of embodiments 121-139, wherein the method activates CB2 on microglia in the brain of the subject.

141. The method of any one of embodiments 121-140, wherein the method reduces activation of microglia in the brain of the subject.

142. The method of any one of embodiments 121-141, wherein the method treats the reticuloendothelial system.

143. The method of any one of embodiments 121-142, wherein the method treats the skeletal system.

144. The method of any one of embodiments 121-143, wherein the method results in reduced inflammation levels in an organ other than the brain.

145. The method of embodiment 121, wherein the organ other than the brain is selected from the liver, spleen, lungs, adrenal gland, kidney, heart, articular cartilage, articular joint space, and bone marrow.

146. The method of any one of embodiments 121-145, wherein the lifespan of the subject is increased as compared to the expected lifespan of an individual with the same lysosomal storage disease or disorder that has not been administered the selective agonist of the CB2 cannabinoid receptor.

147. The method of any one of embodiments 121-146 wherein the method further comprises administering one or more additional therapeutic agents.

148. The method of embodiment 147, wherein the additional therapeutic agent comprises an enzyme replacement therapy, gene therapy, substrate reduction therapy, chaperone therapy, or a small molecule therapy.

149. The method of any one of embodiments 121-148, further comprising administering a direct or indirect agonist of the CB1 cannabinoid receptor.

150. The method of any one of embodiments 1-116 and 121-149, wherein the CB2 agonist results in an improvement in the subject’s myelination.

151. The method of any one of embodiments 1-116 and 121-150, wherein the CB2 agonist inhibits or reduces demyelination in the subject.

152. The method of any one of embodiments 1-116 and 121-151, wherein the CB2 agonist inhibits or reduces astrocyte activation in the subject.

153. A method of evaluating efficacy of a therapy for treating a lysosomal storage disease or disorder in a subject who previously received the therapy, comprising: a) obtaining a post-therapy expression level of CB2 cannabinoid receptor in the subject; and b) comparing the post-therapy expression level to a pre-therapy expression level of CB2 cannabinoid receptor in the subject, wherein a reduced expression level indicates an effective therapy and an elevated expression level indicates an ineffective therapy.

154. The method of embodiment 153, wherein the therapy is an enzyme replacement therapy, gene therapy, bone-marrow transplantation, substrate reduction or inhibition therapy, glucocorticoid, inhibitor of histone deacetylase, chaperone therapy, or any combination thereof.

155. The method of embodiment 153, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

156. The method of embodiment 155, wherein the lipidoses is selected Gaucher disease, Fabry disease, Farber Disease, Krabbe Disease, Metachromatic Leukodystrophy, Acid Spingomyelinase Deficiency (ASMD), Niemann-Pick Disease Type C, Gangliosidoses 1, and Gangliosidoses 2.

157. The method of embodiment 155, wherein the mucopolysaccharidoses is selected from MPS types I, II, III, IV, VI, VII and IX.

158. The method of embodiment 155, wherein the Mucolipidoses is selected from Mucolipidoses I, Mucolipidoses 2, Mucolipidoses 3 and Mucolipidoses 4.

159. The method of embodiment 155, wherein the glycogen storage disease is Pompe Disease.

160. The method of embodiment 155, wherein the carbohydrate storage diseases is selected from Mannosidosis and Sialidosis.

161. The method of embodiment 155, wherein the transport diseases is selected from Cystinosis and Salla Disease.

162. The method of embodiment 155, wherein the neuronal ceroid lipofuscinosis is Batten’s disease.

163. The method of any one of embodiments 153-162, further comprising administering to the subject a selective or specific agonist of the CB2 cannabinoid receptor.

164. The method of embodiment 163, wherein the selective or specific CB2 agonist is administered to the subject as a pharmaceutical composition.

165. The method of embodiment 164, wherein the pharmaceutical composition comprises the selective or specific agonist of the CB2 cannabinoid receptor and one or more pharmaceutically acceptable salts, excipients or vehicles.

166. The method of embodiment 164 or 165, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

167. The method of any one of embodiments 163-166, wherein the selective or specific agonist of the CB2 cannabinoid receptor selected from HU-308, JWH-015, GW-405833 (L-768242), AM-1241, JWH-133, GW-842166X, and the following compounds:

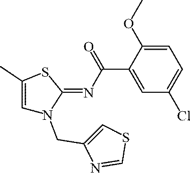 [SMILES: COc1ccc(Cl)cc1C(=O)/N=c1\sc(C)cn1Cc1cscn1]

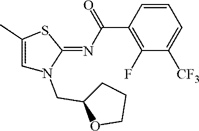 [SMILES: Cc1cn(C[C@H]2CCCO2)/c(=N/C(=O)c2cccc(C(F)(F)F)c2F)s1]

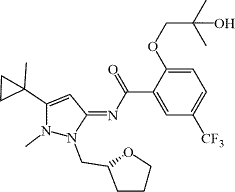

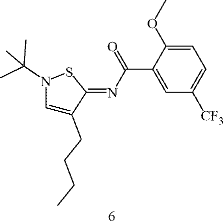

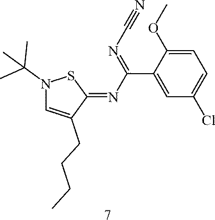

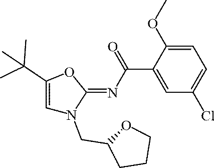

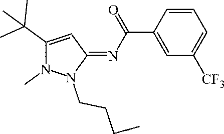 [SMILES: CCCCn1/c(=N/C(=O)c2cccc(C(F)(F)F)c2)cc(C(C)(C)C)n1C]

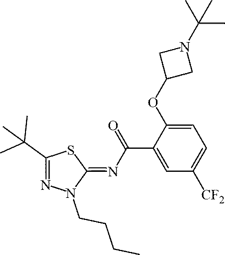

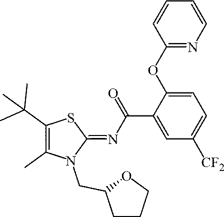

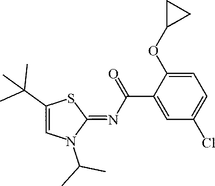 [SMILES: CC(C)n1cc(C(C)(C)C)s/c1=N\C(=O)c1cc(Cl)ccc1OC1CC1]

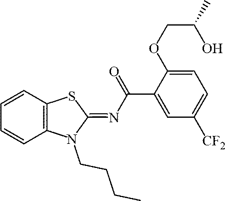

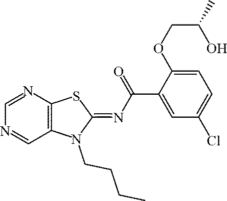 [SMILES: CCCCn1/c(=N/C(=O)c2cc(Cl)ccc2OC[C@H](C)O)sc2ncncc21]

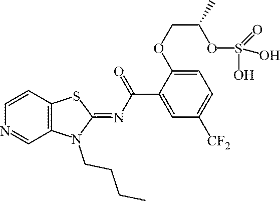

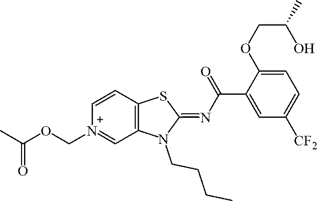

168. The method of any one of embodiments 163-167, wherein the method prevents neuronal death associated with the lysosomal storage disease or disorder.

169. The method of any one of embodiments 163-168, wherein the method improves the subject’s cognition, learning and/or memory.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for reducing inflammation in a subject that has a lysosomal storage disease or disorder comprising, selectively or specifically activating the CB2 cannabinoid receptor in the subject.

2. The method of claim 1, wherein the inflammation is modulated in the subject’s periphery.

3. The method of claim 1, wherein the inflammation is modulated by activating the CB2 receptor on peripheral macrophages in the subject.

4. The method of claim 1, wherein the inflammation is modulated in the subject’s CNS.

5. The method of claim 1, wherein the inflammation is modulated in the subject’s CNS by activating the CB2 receptor on microglia in the subject.

6. The method of claim 1, wherein the inflammation is modulated in the subject’s CNS by inhibiting microglia activation.

7. The method of claim 1, wherein the CB2 cannabinoid receptor is activated by a selective or specific agonist of the CB2 cannabinoid receptor.

8. A method of treating a subject that has a lysosomal storage disease or disorder comprising, administering to the subject a selective or specific agonist of the CB2 cannabinoid receptor.

9. A method of inhibiting a loss of motor skills, cognitive decline, and/or systemic organ pathology associated with a lysosomal storage disease or disorder comprising, administering to the subject a selective or specific agonist of the CB2 cannabinoid receptor.

10. A method of inhibiting macrophage activation in a subject with a lysosomal storage disease or disorder comprising administering to the subject a selective or specific agonist of the CB2 cannabinoid receptor.

11. The method of claim 10, wherein the macrophage is a peripheral macrophage in the subj ect.

12. The method of 10, wherein the macrophage is a microglia in the central nervous system (CNS) of the subject.

13. A method of reducing at least one inflammatory cytokine or chemokine in a subject with a lysosomal storage disease or disorder comprising selectively or specifically activating the CB2 cannabinoid receptor.

14. The method of claim 13, wherein the inflammatory cytokine or chemokine is selected from MCP-1, MIP1 alpha, TNF alpha, IL,-1beta, IL-8, GM-CSF.

15. A method of reducing beta amyloid and or tau protein levels in the brain of subject with a lysosomal storage disease or disorder comprising administering to the subject a selective or specific agonist of the CB2 cannabinoid receptor.

16. A method of evaluating efficacy of a therapy for treating a lysosomal storage disease or disorder in a subject who previously received the therapy, comprising: a) obtaining a post-therapy expression level of CB2 cannabinoid receptor in the subject; and b) comparing the post-therapy expression level to a pre-therapy expression level of CB2 cannabinoid receptor in the subject, wherein a reduced expression level indicates an effective therapy and an elevated expression level indicates an ineffective therapy.

17. The method of claim 16, wherein the therapy is an enzyme replacement therapy, gene therapy, bone-marrow transplantation, substrate reduction or inhibition therapy, glucocorticoid, inhibitor of histone deacetylase, chaperone therapy, or any combination thereof.

18. The method of one of claims 1-17, wherein the lysosomal storage disease or disorder is selected from the group consisting of lipidoses, mucopolysaccharidoses, mucolipidoses, glycogen storage diseases, carbohydrate storage diseases, transport diseases, and neuronal ceroid lipofuscinosis.

19. The method of claim 18, wherein the lipidoses is selected Gaucher disease, Fabry disease, Farber Disease, Krabbe Disease, Metachromatic Leukodystrophy, Acid Spingomyelinase Deficiency (ASMD), Niemann-Pick Disease Type C, Gangliosidoses 1, and Gangliosidoses 2.

20. The method of claim 18, wherein the mucopolysaccharidoses is selected from MPS types I, II, III, IV, VI, VII and IX.

21. The method of claim 18, wherein the Mucolipidoses is selected from Mucolipidoses I, Mucolipidoses 2, Mucolipidoses 3 and Mucolipidoses 4.

22. The method of claim 18, wherein the glycogen storage disease is Pompe Disease.

23. The method of claim 18, wherein the carbohydrate storage diseases is selected from Mannosidosis and Sialidosis.

24. The method of claim 18, wherein the transport diseases is selected from Cystinosis and Salla Disease.

25. The method of claim 18, wherein the neuronal ceroid lipofuscinosis is Batten’s disease.

26. The method of claim 18, wherein the lysosomal storage disease or disorder is Farber Disease, or mucopolysaccharidosis type IIIA (MPS IIIA).

27. The method of any one of claims 7-15, wherein the selective or specific CB2 agonist is administered to the subject as a pharmaceutical composition.

28. The method of claim 27, wherein the pharmaceutical composition comprises the selective or specific agonist of the CB2 cannabinoid receptor and one or more pharmaceutically acceptable salts, excipients or vehicles.

29. The method of claim 27, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.

30. The method of any one of claims 7-15, wherein the selective or specific agonist of the CB2 cannabinoid receptor is selected from HU-308, JWH-015, GW-405833 (L-768242), AM-1241, JWH-133, GW-842166X, and the following compounds: 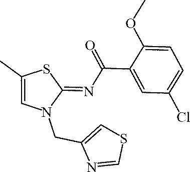 [SMILES: COc1ccc(Cl)cc1C(=O)/N=c1\sc(C)cn1Cc1cscn1] 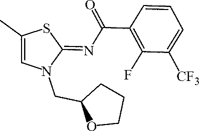 [SMILES: Cc1cn(C[C@H]2CCCO2)/c(=N/C(=O)c2cccc(C(F)(F)F)c2F)s1] 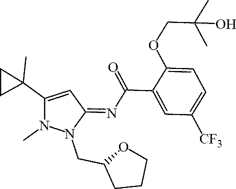 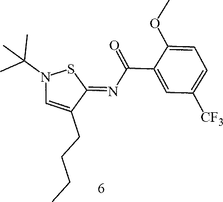 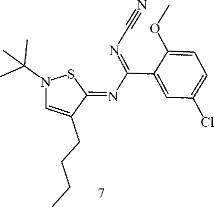 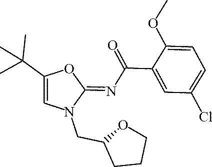 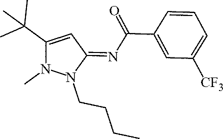 [SMILES: CCCCn1/c(=N/C(=O)c2cccc(C(F)(F)F)c2)cc(C(C)(C)C)n1C] 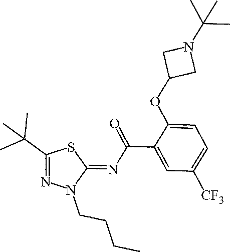 [SMILES: CCCCn1nc(C(C)(C)C)s/c1=N\C(=O)c1cc(C(F)(F)F)ccc1OC1CN(C(C)(C)C)C1] 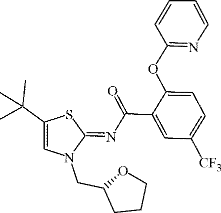 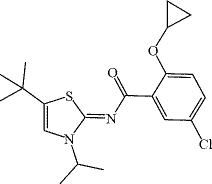 [SMILES: CC(C)n1cc(C(C)(C)C)s/c1=N\C(=O)c1cc(Cl)ccc1OC1CC1] 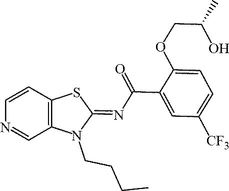 [SMILES: CCCCn1/c(=N/C(=O)c2cc(C(F)(F)F)ccc2OC[C@H](C)O)sc2ccncc21] 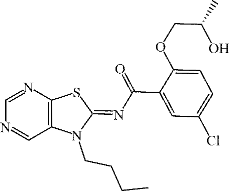 [SMILES: CCCCn1/c(=N/C(=O)c2cc(Cl)ccc2OC[C@H](C)O)sc2ncncc21] 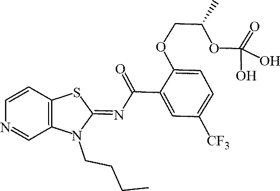 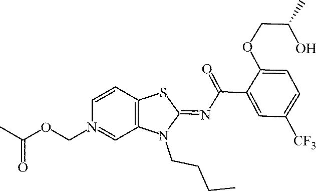
.

31. The method of any one of claims 1-15, wherein the method treats the lysosomal storage disease or disorder, or alleviates one or more symptom associated with the lysosomal storage disease or disorder.

32. The method of any one of claims 1-15, wherein the method prevents neuronal death associated with the lysosomal storage disease or disorder.

33. The method of any one of claims 1-15, wherein the method improves the subject’s cognition, learning and/or memory.

34. The method of any one of claims 1-15, wherein the method reduces inflammation levels in the subject.

35. The method of any one of claims 1-15, wherein the method treats the brain or CNS of the subject.

36. The method of any one of claims 1-15, wherein the method activates CB2 on microglia in the brain of the subject.

37. The method of any one of claims 1-15, wherein the method reduces activation of microglia in the brain of the subject.

38. The method of any one of claims 1-15, wherein the method treats the reticuloendothelial system.

39. The method of any one of claims 1-15, wherein the method treats the skeletal system.

40. The method of any one of claims 1-15, wherein the method results in reduced inflammation levels in an organ other than the brain.

41. The method of claim 40, wherein the organ other than the brain is selected from the liver, spleen, lungs, adrenal gland, kidney, heart, articular cartilage, articular joint space, and bone marrow.

42. The method of any one of claims 1-15, wherein the lifespan of the subject is increased as compared to the expected lifespan of an individual with the same lysosomal storage disease or disorder that has not been administered the selective agonist of the CB2 cannabinoid receptor.

43. The method of any one of claims 1-15 wherein the method further comprises administering one or more additional therapeutic agents.

44. The method of claim 43, wherein the additional therapeutic agent comprises an enzyme replacement therapy, gene therapy, substrate reduction therapy, chaperone therapy, or a small molecule therapy.

45. The method of any one of claims 1-15, further comprising activating the CB1 cannabinoid receptor.

46. The method of claim 45, further comprising administering a direct or indirect agonist of the CB1 cannabinoid receptor, wherein the indirect agonist is optionally a FAAHi.

47. The method of any one of claims 1-15, wherein the CB2 agonist results in an improvement in the subject’s myelination.

48. The method of any one of claims 1-15, wherein the CB2 agonist inhibits or reduces demyelination in the subject.

49. The method of any one of claims 1-15, wherein the CB2 agonist inhibits or reduces astrocyte activation in the subject.

50. A composition comprising an agent capable of selectively activating the CB2 cannabinoid receptor, for use in a method of treating a lysosomal disease or disorder.

51. The composition of claim 50, further comprising an agent capable of directly or indirectly activating the CB1 cannabinoid receptor.

52. A pharmaceutical composition comprising the composition of claim 50 or 51, and one or more pharmaceutically acceptable salts, excipients or vehicles.

53. The pharmaceutical composition of claim 52, wherein the pharmaceutical composition comprises one or more agents selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and /or surfactants.
